(19) United States
(12) Patent Application Publication
(10) Pub. No.: US 2009/0186901 A1
(43) Pub. Date: Jul. 23, 2009

(54) CHEMICAL COMPOUNDS

(76) Inventors: Ulrich Reiser, Vienna (AT); Oliver Kraemer, Vienna (AT); Peter Sennhenn, Muenchen (DE); Walter Spevak, Oberrohrbach (AT)

Correspondence Address: MICHAEL P. MORRIS;BOEHRINGER INGELHEIM USA CORPORATION, 900 RIDGEBURY RD, P. O. BOX 368, RIDGEFIELD, CT, 06877-0368, US

(21) Appl. No.: 12/299,204

(22) PCT Filed: May 3, 2007
(86) PCT No.: PCT/EP2007/054320

§ 371 Date: Dec. 5, 2008

(30) Foreign Application Priority Data
May 5, 2006 (EP) 06113571.1

(51) Int. Cl. A61K 31/499; A61K 31/438; C07D 471/10

(52) U.S. Cl. 514/253.03; 514/278; 544/230; 546/18

(57) ABSTRACT

The present invention relates to new heterocyclic compounds of general formula (1) wherein the groups R¹ to R⁷, k, X and Y have the meanings given in the claims and specification, the isomers and salts thereof as well as the use thereof as medicaments. Background to the invention WO 01/36423 describes 3,4-dihydrospiro[chromene-2,4′-piperidine] derivatives for treating diseases of the central nervous system. Spirocyclic heterocycles as δ-opioid receptor ligands for treating pain and anxiety states as well as diseases of the gastrointestinal tract are known from WO 2005/033073. The aim of the present invention is to indicate new active substances which can be used for the prevention and/or treatment of diseases characterised by excessive or abnormal cell proliferation.

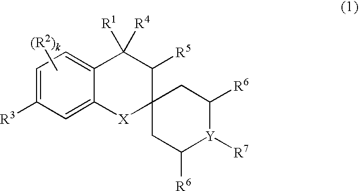

The present invention relates to new heterocyclic compounds of general formula (1)

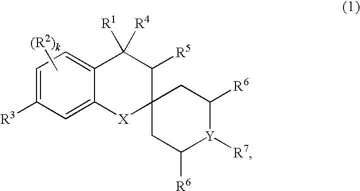

wherein the groups R¹ to R⁷, k, X and Y have the meanings given in the claims and specification, the isomers and salts thereof as well as the use thereof as medicaments.

### BACKGROUND TO THE INVENTION

WO 01/36423 describes 3,4-dihydrospiro[chromene-2,4′-piperidine] derivatives for treating diseases of the central nervous system. Spirocyclic heterocycles as δ-opioid receptor ligands for treating pain and anxiety states as well as diseases of the gastrointestinal tract are known from WO 2005/033073.

The aim of the present invention is to indicate new active substances which can be used for the prevention and/or treatment of diseases characterised by excessive or abnormal cell proliferation.

### DETAILED DESCRIPTION OF THE INVENTION

Surprisingly, it has been found that compounds of general formula (1), wherein groups R¹ to R⁷, k, X and Y have the meanings given hereinafter, act as inhibitors of specific cell cycle enzymes. Thus the compounds according to the invention may be used for example for the treatment of diseases connected with the activity of specific cell cycle enzymes and characterised by excessive or abnormal cell proliferation.

The present invention therefore relates to compounds of general formula (1)

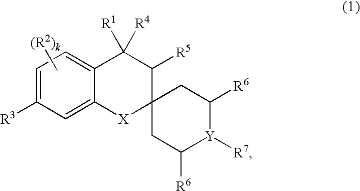

wherein
X denotes —O—, —S—, —SO— or —SO₂— and
Y denotes N or CH,
R¹ is selected from among C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, all the above-mentioned groups optionally being substituted by one or more identical or different R^{a} and/or R^{b},
R² and R³ are each independently of one another selected from among R^{a} and R^{b}, or
R³ together with an adjacent R² in the ortho position and the two carbon atoms to which R² and R³ are fixed, may form a phenyl ring, a 5-6 membered heteroaryl, 5-7 membered cycloalkyl or 5-7 membered heterocycloalkyl, while the above-mentioned ring systems may optionally be substituted by one or more identical or different R^{a} and/or R^{b},
R⁴ denotes hydrogen, C₁₋₆alkyl or C₁₋₆haloalkyl, optionally substituted by one or more identical or different groups —OR^{h} and/or —NR^{h}R^{h},
R⁵ is selected from among hydrogen, C₁₋₆haloalkyl, halogen, —CN, —C(O)OR^{h}, —C(O)NR^{h}R^{h} and C₁₋₆alkyl, the latter optionally being substituted by one or more identical or different groups —OR^{h},
each R⁶ is selected independently of one another from among hydrogen, C₁₋₆alkyl, C₆₋₁₀aryl and halogen,
R⁷ in the event that Y denotes N, is selected from among hydrogen, R^{a}, —OR^{a}, —NR^{a}R^{a}, —S(O)R^{a}, —S(O)NR^{a}R^{a}, —S(O)₂R^{a}, —S(O)₂OR^{a}, —S(O)₂NR^{a}R^{a}, —[S(O)₂]₂R^{a}, —S(O)OR^{a}, —C(O)R^{a}, —C(S)R^{a}, —N(R^{g})C(O)R^{a}, —C(NOH)R^{a}, —C(NR^{g})R^{a}, —C(O)OR^{a}, —C(O)SR^{a}, —C(O)NR^{a}R^{a}, —C(S)NR^{a}R^{a}, —C(O)N(R^{g})NR^{a}R^{a}, —N(R^{g})C(O)NR^{a}R^{a}, —C(NR^{g})OR^{a}, —C(NR^{g})SR^{a}, —C(NR^{g})NR^{a}R^{a}, —C(O)N(R^{g})C(O)R^{a}, —[C(O)]₂R^{a}, —[C(O)]₂OR^{a}, —[C(O)]₂NR^{a}R^{a} and —C(O)N(R^{g})C(O)OR^{a}, or

R⁷, in the event that Y denotes CH, is selected from among 2-6 membered heteroalkyl, 5-12 membered heteroaryl, 3-14 membered heterocycloalkyl, all the above-mentioned groups optionally being substituted by one or more identical or different R^{a} and/or R^{b}, as well as —NR^{a}R^{a}, —N(OR^{a})R^{a}, —N(R^{g})NR^{a}R^{a}, —N(R^{g})S(O)R^{a}, —N(R^{g})S(O)₂R^{a}, —N[S(O)₂R^{a}]₂, —N(R^{g})S(O)₂OR^{a}, —N(R^{g})S(O)₂NR^{a}R^{a}, —N(R^{g})S(O)OR^{a}, —N(R^{g})S(O)NR^{a}R^{a}, —N(R^{g})C(O)R^{a}, —N[C(O)R^{a}]₂, —N(R^{g})C(S)R^{a}, —N[C(O)R^{a}]NR^{a}R^{a}, —N(R^{g})N(R^{g})C(O)R^{a}, —N(OR^{g})C(O)R^{a}, —N(R^{g})C(NOH)R^{a}, —N(R^{g})C(NR^{g})R^{a}, —N(R^{g})C(O)OR^{a}, —N(R^{g})C(O)SR^{a}, —N(R^{g})C(O)NR^{a}R^{a}, —N(R^{g})C(S)NR^{a}R^{a}, —N(R^{g})C(O)NR^{g}NR^{a}R^{a}, N(R^{g})N(R^{g})C(O)NR^{a}R^{a}, —N(R^{g})C(NR^{g})OR^{a}, —N(R^{g})C(NR^{g})SR^{a}, —N(R^{g})C(NR^{g})NR^{a}R^{a}, [N(R^{g})C(O)]₂R^{a}, —N(R^{g})[C(O)]₂R^{a}, —N{[C(O)]₂R^{a} }₂, —N(R^{g})[C(O)]₂OR^{a}, —N(R^{g})[C(O)]₂NR^{a}R^{a}, —N {[C(O)]₂OR^{a}}₂, —N {[C(O)]₂NR^{a}R^{a}}₂ and —[N(R^{g})C(O)]₂OR^{a},

k denotes either 0, 1, 2 or 3,
each R^{a} independently of one another denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
each R^{b} denotes a suitable group and is selected independently of one another from among ═O, —OR^{c}, C₁₋₃haloalkyloxy, —OCF₃, ═S, —SR^{c}, ═NR^{c}, ═NOR^{c}, ═NNR^{c}R^{c}, ═NN(R^{g})C(O)NR^{c}R^{c}, NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)R^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N {[C(O)]₂R^{c}}₂, —N(R^{g})[C(O)]₂OR^{c}, —N(R^{g})[C(O)]₂NR^{c}R^{c}, —N {[C(O)]₂OR^{c}}₂, —N {[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c},
each R^{c} independently of one another denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
each R^{d} denotes a suitable group and is selected independently of one another from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}, —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R^{g})C(NR^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e}, —N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g})[C(O)]₂R^{e}, —N{[C(O)]₂R^{e} }₂, —N(R^{g})[C(O)]₂OR^{e}, —N(R^{g})[C(O)]₂NR^{e}R^{e}, —N{[C(O)]₂OR^{e} }₂, —N {[C(O)]₂NR^{e}R^{e} }₂, —[N(R^{g})C(O)]₂R^{e}, —N(R^{g})C(NR^{g})OR^{e}, —N(R^{g})C(NOH)R^{e}, —N(R^{g})C(NR^{g})SR^{e} and —N(R^{g})C(NR^{g})NR^{e}R^{e},
each R^{e} independently of one another denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{f} denotes a suitable group and in each case is selected independently of one another from among ═O, —OR^{g}, C₁₋₃haloalkyloxy, —OCF₃, ═S, —SR^{g}, ═NR^{g}, ═NOR^{g}, ═NNR^{g}R^{g}, ═NN(R^{h})C(O)NR^{g}R^{g}, —NR^{g}R^{g}, —ONR^{g}R^{g}, —N(R^{h})NR^{g}R^{g}, halogen, —CF₃, —CN, —NC, —OCN, —SCN, —NO, —NO₂, ═N₂, —N₃, —S(O)R^{g}, —S(O)OR^{g}, —S(O)₂R^{g}, —S(O)₂OR^{g}, —S(O)NR^{g}R^{g}, —S(O)₂NR^{g}R^{g}, —OS(O)R^{g}, —OS(O)₂R^{g}, —OS(O)₂OR^{g}, —OS(O)NR^{g}R^{g}, —OS(O)₂NR^{g}R^{g}, —C(O)R^{g}, —C(O)OR^{g}, —C(O)SR^{g}, —C(O)NR^{g}R^{g}, —C(O)N(R^{h})NR^{g}R^{g}, —C(O)N(R^{h})OR^{g}, —C(NR^{h})NR^{g}R^{g}, —C(NOH)R^{g}, —C(NOH)NR^{g}R^{g}, —OC(O)R^{g}, —OC(O)OR^{g}, —OC(O)SR^{g}, —OC(O)NR^{g}R^{g}, —OC(NR^{h})NR^{g}R^{g}, —SC(O)R^{g}, —SC(O)OR^{g}, —SC(O)NR^{g}R^{g}, —SC(NR^{h})NR^{g}R^{g}, —N(R^{h})C(O)R^{g}, —N[C(O)R^{g}]₂, —N(OR^{h})C(O)R^{g}, —N(R^{h})C(NR^{h})R^{g}, —N(R^{h})N(R^{h})C(O)R^{g}, —N[C(O)R^{g}]NR^{g}R^{g}, —N(R^{h})C(S)R^{g}, —N(R^{h})S(O)R^{g}, —N(R)S(O)OR^{g}, —N(R^{h})S(O)₂R^{g}, —N[S(O)₂R^{g}]₂, —N(R^{h})S(O)₂OR^{g}, —N(R^{h})S(O)₂NR^{g}R^{g}, —N(R^{h}) [S(O)₂]₂R^{g}, —N(R^{h})C(O)OR^{g}, —N(R^{h})C(O)SR^{g}, —N(R^{h})C(O)NR^{g}R^{g}, —N(O)C(O)NR^{h}NR^{g}R^{g}, —N (1)N(R^{h})C(O)NR^{g}R^{g}, —N(R^{h})C(S)NR^{g}R^{g}, —[N(R^{h})C(O)]₂R^{g}, —N(R^{h})[C(O)]₂R^{g}, —N {[C(O)]₂R^{g} }₂, —N(R^{h})[C(O)]₂OR^{g}, —N(R^{h})[C(O)]₂NR^{g}R^{g}, —N {[C(O)]₂OR^{g}}₂, —N {[C(O)]₂NR^{g}R^{g}}₂, —[N(R^{h})C(O)]₂OR^{g}, —N(R^{h})C(NR^{h})OR^{g}, —N(R^{h})C(NOH)R^{g}, —N(R^{h})C(NR^{h})SR^{g} and —N(R^{h})C(NR^{h})NR^{g}R^{g},
each R^{g} independently of one another denotes hydrogen or a group optionally substituted by one or more identical or different R^{h}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
each R^{h} is selected independently of one another from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
optionally in the form of the tautomers, the racemates, the enantiomers, the diastereomers and the mixtures thereof, and optionally the pharmacologically acceptable salts thereof,
with the proviso that the compounds
4-(7,8-dihydro-6H-[1,3]dioxolo[4,5-g]spiro[chromene-2,1′-cyclohexan]-8-yl)-methoxyphenyl;
methyl-1-(3,4-dihydrospiro[chromene-2,1′-cyclohexan]-4-yl)-1H-imidazole-5-carboxylate;
ethyl-1-(5-chloro-7-methoxy-3,4-dihydrospiro[chromene-2,1′-cyclohexan]-4-yl)-1H-imidazole-5-carboxylate;
ethyl-1-(5-chloro-7-methoxy-3,4-dihydrospiro[chromene-2,1′-cyclohexan]-4-yl)-2-mercapto-1H-imidazole-5-carboxylate;
methyl-1-(3,4-dihydrospiro[chromene-2,1′-cyclohexan]-4-yl)-2-mercapto-1H-imidazole-5-carboxylate;
1-(3,4-dihydrospiro[chromene-2,1′-cyclohexan]-4-yl)-1H-imidazole-5-carboxylic acid;
ethyl-1-(3′,4′-dihydrospiro[cyclohexane-1,2′-thiochromen]-4′-yl)-1H-imidazole-5-carboxylate;
ethyl-1-(3′,4′-dihydrospiro[cyclohexane-1,2′-thiochromen]-4′-yl)-2-mercapto-1H-imidazole-5-carboxylate;
ethyl-1-(1′-oxido-3′,4′-dihydrospiro[cyclohexane-1,2′-thiochromen]-4′-yl)-1H-imidazole-5-carboxylate;
ethyl-2-mercapto-1-(1′-oxido-3′,4′-dihydrospiro[cyclohexane-1,2′-thiochromen]-4′-yl)-1H-imidazole-5-carboxylate;
ethyl-1-(1′, 1′-dioxido-3′,4′-dihydrospiro[cyclohexane-1,2′-thiochromen]-4′-yl)-1H-imidazole-5-carboxylate;
ethyl-1-(1′, 1′-dioxido-3′,4′-dihydrospiro[cyclohexane-1,2′-thiochromen]-4′-yl)-2-mercapto-1H-imidazole-5-carboxylate;
1-(3,4-dihydrospiro[chromene-2,1′-cyclohexan]-4-yl)-5-(methoxycarbonyl)-3-methyl-1H-imidazol-3-ium;
methyl-1-oxy-3-(3,4-dihydrospiro[chromene-2,1′-cyclohexan]-4-yl)-3H-imidazol-4-carboxylate;
4-(3,4-dihydrospiro[chromene-2,4′-piperidin]-4-ylmethyl)-N,N-diethylbenzamide;
3-hydroxy-4-(6-fluoro-3,4-dihydrospiro[chromene-2,4′-piperidin]-4-yl)-N,N-diethyl-benzamide;
3-hydroxy-4-(3,4-dihydrospiro[chromene-2,4′-piperidin]-4-yl)-N,N-diethylbenzamide;
5-(5-methoxy-3,4-dihydrospiro[chromene-2,4′-piperidin]-4-yl)-N,N-diethylpyridine-2-carboxamide;
4-(5-methoxy-3,4-dihydrospiro[chromene-2,4′-piperidin]-4-yl)-N,N-diethylbenzamide;
4-(3,4-dihydrospiro[chromene-2,4′-piperidin]-4-yl)-N,N-diethylbenzamide;
4-(6-fluoro-3,4-dihydrospiro[chromene-2,4′-piperidin]-4-yl)-N,N-diethylbenzamide;
4-(6-cyclopropylmethoxy-3,4-dihydrospiro[chromene-2,4′-piperidin]-4-yl)-N,N-diethylbenzamide;
5-(3,4-dihydrospiro[chromene-2,4′-piperidin]-4-yl)-N,N-diethylpyridine-2-carboxamide;
5-(6-fluoro-3,4-dihydrospiro[chromene-2,4′-piperidin]-4-yl)-N,N-diethylpyridine-2-carboxamide;
5-(6,7-dimethyl-3,4-dihydrospiro[chromene-2,4′-piperidin]-4-yl)-N,N-diethylpyridine-2-carboxamide;
4-(6-hydroxy-3,4-dihydrospiro[chromene-2,4′-piperidin]-4-yl)-N,N-diethylbenzamide;
4-(5-hydroxy-3,4-dihydrospiro[chromene-2,4′-piperidin]-4-yl)-N,N-diethylbenzamide;
4-(6-methyl-3,4-dihydrospiro[chromene-2,4′-piperidin]-4-yl)-N,N-diethylbenzamide;
{3,4-dihydro-4-(4-methylphenyl)spiro[chromene-2,4′-piperidin]-1′-yl}-acetic acid;
ethyl-{3,4-dihydro-4-(4-methylphenyl)spiro[chromene-2,4′-piperidin]-1′-yl}-acetate;
{3,4-dihydro-4-(4-fluorophenyl)spiro[chromene-2,4′-piperidin]-1′-yl}-acetic acid;
ethyl-{3,4-dihydro-4-(4-fluorophenyl)spiro[chromene-2,4′-piperidin]-1′-yl}-acetate and the compounds with the structures (i) and (ii)

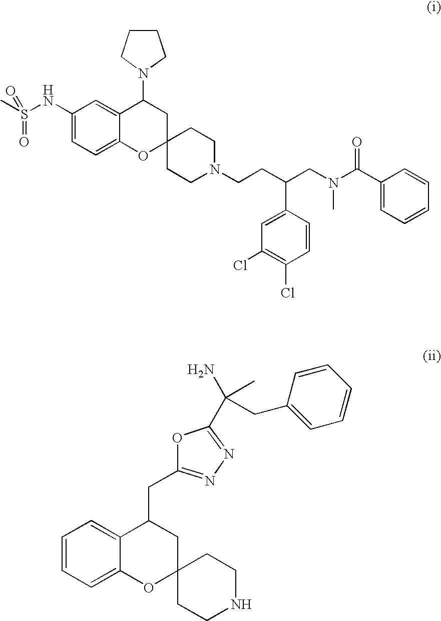

are excluded.

In one aspect the invention relates to compounds wherein

X denotes oxygen and
R⁵ and R⁶ denote hydrogen.

In another aspect the invention relates to compounds wherein

R¹ denotes C₆₋₁₀aryl or 5-12 membered heteroaryl, optionally substituted by one or more identical or different R^{a} and/or R^{b}, and
R^{a} and R^{b} are as hereinbefore defined.

In another aspect the invention relates to compounds, wherein

in the event that R¹ is substituted by one or two R^{b} and no R^{a}, none of these R^{b} may be —C(O)NR^{c}R^{c} and
R¹, R^{a} and R^{c} are as hereinbefore defined.

In another aspect the invention relates to compounds, wherein

Y denotes nitrogen.

In another aspect the invention relates to compounds, wherein

R¹ denotes C₆₋₁₀aryl or 5-12 membered heteroaryl, optionally substituted by one or more identical or different groups, selected from among —OR^{c} and halogen, and
R⁴ denotes hydrogen and
R^{c} is as hereinbefore defined.

In another aspect the invention relates to compounds, wherein

R³ is not hydrogen.

In another aspect the invention relates to compounds, wherein

R³ is selected from among —OR^{c}, —NR^{c}R^{c} and 3-14 membered heterocycloalkyl, the latter optionally being substituted by one or more identical or different R^{b} and/or R^{c} and
R^{b} and R^{c} are as hereinbefore defined.

In another aspect the invention relates to compounds of general formula (2)

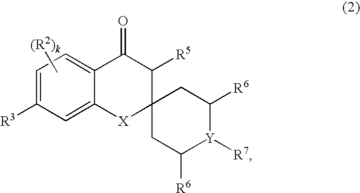

which are suitable as intermediate products for preparing compounds of general formula (1) and wherein R², R⁵ to R⁷, X, Y and k have the meanings given for formula (1) and R³ has one of the meanings given for formula (1) other than hydrogen, which may also be an object of the invention.

In another aspect the invention relates to compounds—or the pharmacologically acceptable salts thereof—of general formula (1) as medicaments.

In another aspect the invention relates to pharmaceutical preparations, containing as active substance one or more compounds of general formula (1)—or the pharmacologically acceptable salts thereof—optionally in combination with conventional excipients and/or carriers.

In another aspect the invention relates to the use of compounds of general formula (1)

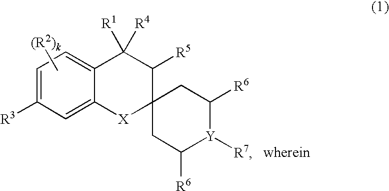

X denotes-O—, —S—, —SO— or —SO₂— and
Y denotes N or CH,
R¹ is selected from among C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, all the above-mentioned groups optionally being substituted by one or more identical or different R^{a} and/or R^{b},
R² and R³ are each independently of one another selected from among R^{a} and R^{b}, or
R³ together with an adjacent R² in the ortho position and the two carbon atoms to which R² and R³ are fixed, may form a phenyl ring, a 5-6 membered heteroaryl, 5-7 membered cycloalkyl or 5-7 membered heterocycloalkyl, while the above-mentioned ring systems may optionally be substituted by one or more identical or different R^{a} and/or R^{b},
R⁴ denotes hydrogen, C₁₋₆alkyl or C₁₋₆haloalkyl, optionally substituted by one or more identical or different groups —OR^{h} and/or —NR^{h}R^{h},
R⁵ is selected from among hydrogen, C₁₋₆haloalkyl, halogen, —CN, —C(O)OR^{h}, —C(O)NR^{h}R^{h} and C₁₋₆alkyl, the latter optionally being substituted by one or more identical or different groups —OR^{h},
each R⁶ is selected independently of one another from among hydrogen, C₁₋₆alkyl, C₆₋₁₀aryl and halogen,
R⁷, in the event that Y denotes N, is selected from among hydrogen, R^{a}, —OR^{a}, —NR^{a}R^{a}, —S(O)R^{a}, —S(O)NR^{a}R^{a}, —S(O)₂R^{a}, —S(O)₂OR^{a}, —S(O)₂NR^{a}R^{a}, —[S(O)₂]₂R^{a}, —S(O)OR^{a}, —C(O)R^{a}, —C(S)R^{a}, —N(R^{g})C(O)R^{a}, —C(NOH)R^{a}, —C(NR^{g})R^{a}, —C(O)OR^{a}, —C(O)SR^{a}, —C(O)NR^{a}R^{a}, —C(S)NR^{a}R^{a}, —C(O)N(R^{g})NR^{a}R^{a}, —N(R^{g})C(O)NR^{a}R^{a}, —C(NR^{g})OR^{a}, —C(NR^{g})SR^{a}, —C(NR^{g})NR^{a}R^{a}, —C(O)N(R—)C(O)R^{a}, —[C(O)]₂R^{a}, —[C(O)]₂OR^{a}, —[C(O)]₂NR^{a}R^{a} and —C(O)N(R^{g})C(O)OR^{a}, or
R⁷, in the event that Y denotes CH, is selected from among 2-6 membered heteroalkyl, 5-12 membered heteroaryl, 3-14 membered heterocycloalkyl, all the above-mentioned groups optionally being substituted by one or more identical or different R^{a} and/or R^{b}, as well as —NR^{a}R^{a}, —N(OR^{a})R^{a}, —N(R^{g})NR^{a}R^{a}, —N(R^{g})S(O)R^{a}, —N(R^{g})S(O)₂R^{a}, —N[S(O)₂R^{a}]₂, —N(R^{g})S(O)₂OR^{a}, —N(R^{g})S(O)₂NR^{a}R^{a}, —N(R^{g})S(O)OR^{a}, —N(R^{g})S(O)NR^{a}R^{a}, —N(R^{g})C(O)R^{a}, —N[C(O)R^{a}]₂, —N(R^{g})C(S)R^{a}, —N[C(O)R^{a}]NR^{a}R^{a}, —N(R^{g})N(R^{g})C(O)R^{a}, —N(OR^{g})C(O)R^{a}, —N(R^{g})C(NOH)R^{a}, —N(R^{g})C(NR^{g})R^{a}, —N(R^{g})C(O)OR^{a}, —N(R^{g})C(O)SR^{a}, —N(R^{g})C(O)NR^{a}R^{a}, —N(R^{g})C(S)NR^{a}R^{a}, —N(R^{g})C(O)NR^{g}NR^{a}R^{a}, N(R^{g})N(R^{g})C(O)NR^{a}R^{a}, —N(R^{g})C(NR^{g})OR^{a}, —N(R^{g})C(NR^{g})SR^{a}, —N(R^{g})C(NR^{g})NR^{a}R^{a}, —[N(R^{g})C(O)]₂R^{a}, —N(R^{g})[C(O)]₂R^{a}, —N{[C(O)]₂R^{a}}₂, —N(R^{g})[C(O)]₂OR^{a}, —N(R^{g})[C(O)]₂NR^{a}R^{a}, —N {[C(O)]₂OR^{a} }₂, —N {[C(O)]₂NR^{a}R^{a}}₂ and —[N(R^{g})C(O)]₂OR^{a},
k denotes either 0, 1, 2 or 3,
each R^{a} independently of one another denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{b} denotes a suitable group and is selected independently of one another from among ═O, —OR^{c}, C₁₋₃haloalkyloxy, —OCF₃, ═S, —SR^{c}, ═NR^{c}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}R^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g})[S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N {[C(O)]₂R^{c} }₂, —N(R^{g})[C(O)]₂OR^{c}, —N(R^{g})[C(O)]₂NR^{c}R^{c}, —N {[C(O)]₂OR^{c}}₂, —N {[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c},
each R^{c} independently of one another denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
each R^{d} denotes a suitable group and is selected independently of one another from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}, —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e}, —OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R^{g})C(NR^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e}, —N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g})[C(O)]₂R^{e}, —N{[C(O)]₂R^{e} }₂, —N(R^{g})[C(O)]₂OR^{e}, —N(R^{g})[C(O)]₂NR^{e}R^{e}, —N{[C(O)]₂OR^{e} }₂, —N {[C(O)]₂NR^{e}R^{e} }₂, —[N(R^{g})C(O)]₂OR^{e}, —N(R^{g})C(NR^{g})OR^{e}, —N(R^{g})C(NOH)R^{e}, —N(R^{g})C(NR^{g})SR^{e} and —N(R^{g})C(NR^{g})NR^{e}R^{e},
each R^{e} independently of one another denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{f} denotes a suitable group and is selected independently of one another from among ═O, —OR^{g}, C₁₋₃haloalkyloxy, —OCF₃, ═S, —SR^{g}, ═NR^{g}, ═NOR^{g}, ═NNR^{g}R^{g}, ═NN(R^{h})C(O)NR^{g}R^{g}, —NR^{g}R^{g}, —ONR^{g}R^{g}, —N(R^{h})NR^{g}R^{g}, halogen, —CF₃, —CN, —NC, —OCN, —SCN, —NO, —NO₂, ═N₂, —N₃, —S(O)R^{g}, —S(O)OR^{g}, —S(O)₂R^{g}, —S(O)₂OR^{g}, —S(O)NR^{g}R^{g}, —S(O)₂NR^{g}R^{g}, —OS(O)R^{g}, —OS(O)₂R^{g}, —OS(O)₂OR^{g}, —OS(O)NR^{g}R^{g}, —OS(O)₂NR^{g}R^{g}, —C(O)R^{g}, —C(O)OR^{g}, —C(O)SR^{g}, —C(O)NR^{g}R^{g}, —C(O)N(R^{h})NR^{g}R^{g}, —C(O)N(R^{h})OR^{g}, —C(NR^{h})NR^{g}R^{g}, —C(NOH)R^{g}, —C(NOH)NR^{g}R^{g}, —OC(O)R^{g}, —OC(O)OR^{g}, —OC(O)SR^{g}, —OC(O)NR^{g}R^{g}, —OC(NR^{h})NR^{g}R^{g}, —SC(O)R^{g}, —SC(O)OR^{g}, —SC(O)NR^{g}R^{g}, —SC(NR^{h})NR^{g}R^{g}, —N(R^{h})C(O)R^{g}, —N[C(O)R^{g}]₂, —N(OR^{h})C(O)R^{g}, —N(R^{h})C(NR^{h})R^{g}, —N(R^{h})N(R^{h})C(O)R^{g}, —N[C(O)R^{g}]NR^{g}R^{g}, —N(R^{h})C(S)R^{g}, —N(R^{h})S(O)R^{g}, —N(R^{h})S(O)OR^{g}, —N(R^{h})S(O)₂R^{g}, —N[S(O)₂R^{g}]₂, —N(R^{h})S(O)₂OR^{g}, —N(R^{h})S(O)₂NR^{g}R^{g}, —N(R^{h}) [S(O)₂]₂R^{g}, —N(R^{h})C(O)OR^{g}, —N(R^{h})C(O)SR^{g}, —N(R^{h})C(O)NR^{g}R^{g}, —N(R^{h})C(O)NR^{h}NR^{g}R^{g}, —N(R^{h})N(R^{h})C(O)NR^{g}R^{g}, —N(R^{h})C(S)NR^{g}R^{g}, —[N(R^{h})C(O)]₂R^{g}, —N(R^{h})[C(O)]₂R^{g}, —N {[C(O)]₂R^{g}}₂, —N(R^{h})[C(O)]₂OR^{g}, —N(R^{h})[C(O)]₂NR^{g}R^{g}, —N {[C(O)]₂OR^{g}}₂, —N {[C(O)]₂NR^{g}R^{g}}₂, —[N(R^{h})C(O)]₂OR^{g}, —N(R^{h})C(NR^{h})OR^{g}, —N(R^{h})C(NOH)R^{g}, —N(R^{h})C(NR^{h})SR^{g} and —N(R^{h})C(NR^{h})NR^{g}R^{g},
each R^{g} independently of one another denotes hydrogen or a group optionally substituted by one or more identical or different R^{h}, selected from among C₁₋₆alkyl, 2-6 membered heteroalkyl, C₁₋₁₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{h} is selected independently of one another from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
optionally in the form of the tautomers, the racemates, the enantiomers, the diastereomers and the mixtures thereof, and optionally the pharmacologically acceptable salts thereof,
for preparing a pharmaceutical composition for the treatment and/or prevention of cancer and infectious diseases.

In another aspect the invention relates to the use of compounds of general formula (1) for preparing a medicament for the treatment and/or prevention of cancer, infectious, inflammatory and autoimmune diseases.

In another aspect the invention relates to a pharmaceutical preparation comprising a compound of general formula (1) and at least one other cytostatic or cytotoxic active substance, different from formula (1), optionally in the form of the tautomers, the racemates, the enantiomers, the diastereomers and the mixtures thereof, and optionally the pharmacologically acceptable salts thereof.

(A) Aspects relating to R¹:
(A1) In one aspect the invention relates to compounds of general formula (1), wherein R¹ is selected from among phenyl, naphthyl, biphenyl, pyridyl, thienyl and 1,3-benzodioxolyl, all the above-mentioned groups optionally being substituted by one or more identical or different R^{a} and/or R^{b}.
(A2) In another aspect the invention relates to compounds of general formula (1), wherein R¹ denotes phenyl, optionally substituted by one or two substituents selected from among hydroxy, methyl, ethyl, hydroxymethyl, amino, N,N-dimethylamino, carboxy and halogen.
(A3) In another aspect the invention relates to compounds of general formula (1), wherein R¹ denotes mono- or di-, ortho- and/or para-substituted phenyl.
(A4) In another aspect the invention relates to compounds of general formula (1), wherein R¹ corresponds to 3-hydroxyphenyl.
(B) Aspects relating to R³:
(B1) In one aspect the invention relates to compounds of general formula (1), wherein R³ corresponds to the group —NR⁸R⁹ and R⁸ and R⁹ together with the nitrogen atom to which they are bound form a 5-9 membered heterocycloalkyl, and this may optionally also contain a further heteroatom selected from among nitrogen and oxygen and may be substituted by a methyl, N,N-dimethylamino or cyano group or by the group —NHC(O)Me.
(B2) In another aspect the invention relates to compounds of general formula (1), wherein R³ corresponds to the group —NR⁸R⁹, where R⁸ denotes a hydrogen atom or a methyl group and R⁹ corresponds to a benzyl, phenyl or cyclopentyl group or a haloalkyl.
(B3) In another aspect the invention relates to compounds of general formula (1), wherein R³ denotes the groups hydroxy, methyl, phenyl, pyridyl, methoxy, ethoxy, propoxy, isopropoxy, 3-methylbutoxy, isobutoxy, 2-methylallyloxy, allyloxy, but-2-enyloxy, but-2-ynyloxy, prop-2-ynyloxy, 2-methoxy-ethoxy, cyclobutoxy, cyclopentyl, cyclopentyloxy, cyclohexyloxy, cycloheptyloxy, cyclopropylmethoxy, cyclohexylmethoxy, benzyloxy, the latter optionally being substituted at the phenyl ring by an isopropyl, cyanomethoxy, azetidinoxy, pyrrolidinoxy or tetrahydrofuranoxy group, the latter optionally being substituted by an oxo and/or methyl group.
(B4) In another aspect the invention relates to compounds of general formula (1), wherein R³ has the partial structure (iii)

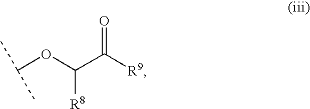

R⁸ denotes hydrogen or methyl and R⁹ denotes tert.-butyl, methyl, thienyl, methoxy, tert.-butoxy or amino.
(B5) In another aspect the invention relates to compounds of general formula (1), wherein R³ together with an adjacent R² in the ortho position and the two carbon atoms to which R² and R³ are fixed, forms a phenyl ring or R² and R³ together form an ethylenedioxy bridge.

All the above-mentioned aspects A1-A4 may be combined with all the above-mentioned aspects B1-B5 to form preferred embodiments of the compounds according to the invention.

### DEFINITIONS

As used herein, the following definitions apply, unless stated otherwise:

The use of the prefix C_{x-y}, wherein x and y in each case represent a natural number (x<y), indicates that the chain or ring structure or combination of chain and ring structure specified and mentioned in direct conjunction may consist of a total of at most y and at least x carbon atoms.

Alkyl is made up of the sub-groups saturated hydrocarbon chains and unsaturated hydrocarbon chains, while the latter may be further subdivided into hydrocarbon chains with a double bond (alkenyl) and hydrocarbon chains with a triple bond (alkynyl). Alkenyl contains at least one double bond, alkynyl contains at least one triple bond. If a hydrocarbon chain were to carry both at least one double bond and also at least one triple bond, by definition it would belong to the alkynyl sub-group. All the sub-groups mentioned above may further be divided into straight-chain (unbranched) and branched. If an alkyl is substituted, the substitution may be mono- or polysubstitution in each case, at all the hydrogen-carrying carbon atoms, independently of one another.

Examples of representatives of individual sub-groups are listed below.

### Straight-Chain (Unbranched) or Branched Saturated Hydrocarbon Chains:

methyl; ethyl; n-propyl; isopropyl (1-methylethyl); n-butyl; 1-methylpropyl; isobutyl (2-methylpropyl); sec.-butyl (1-methylpropyl); tert.-butyl (1,1-dimethylethyl); n-pentyl; 1-methylbutyl; 1-ethylpropyl; isopentyl (3-methylbutyl); neopentyl (2,2-dimethyl-propyl); n-hexyl; 2,3-dimethylbutyl; 2,2-dimethylbutyl; 3,3-dimethylbutyl; 2-methyl-pentyl; 3-methylpentyl; n-heptyl; 2-methylhexyl; 3-methylhexyl; 2,2-dimethylpentyl; 2,3-dimethylpentyl; 2,4-dimethylpentyl; 3,3-dimethylpentyl; 2,2,3-trimethylbutyl; 3-ethylpentyl; n-octyl; n-nonyl; n-decyl etc.

### Straight-Chain (Unbranched) or Branched Alkenyl:

vinyl(ethenyl); prop-1-enyl; allyl(prop-2-enyl); isopropenyl; but-1-enyl; but-2-enyl; but-3-enyl; 2-methyl-prop-2-enyl; 2-methyl-prop-1-enyl; 1-methyl-prop-2-enyl; 1-methyl-prop-1-enyl; 1-methylidenepropyl; pent-1-enyl; pent-2-enyl; pent-3-enyl; pent-4-enyl; 3-methyl-but-3-enyl; 3-methyl-but-2-enyl; 3-methyl-but-1-enyl; hex-1-enyl; hex-2-enyl; hex-3-enyl; hex-4-enyl; hex-5-enyl; 2,3-dimethyl-but-3-enyl; 2,3-dimethyl-but-2-enyl; 2-methylidene-3-methylbutyl; 2,3-dimethyl-but-1-enyl; hexa-1,3-dienyl; hexa-1,4-dienyl; penta-1,4-dienyl; penta-1,3-dienyl; buta-1,3-dienyl; 2,3-dimethylbuta-1,3-diene etc.

### Straight-Chain (Unbranched) or Branched Alkynyl:

ethynyl; prop-1-ynyl; prop-2-ynyl; but-1-ynyl; but-2-ynyl; but-3-ynyl; 1-methyl-prop-2-ynyl etc.

By the terms propyl, butyl, pentyl, hexyl, heptyl, octyl, nonyl, decyl etc. without any further definition are meant saturated hydrocarbon groups with the corresponding number of carbon atoms, all the isomeric forms being included.

By the terms propenyl, butenyl, pentenyl, hexenyl, heptenyl, octenyl, nonenyl, decenyl etc. without any further definition are meant unsaturated hydrocarbon groups with the corresponding number of carbon atoms and a double bond, all the isomeric forms, i.e. (Z)/(E) isomers, being included where applicable.

By the terms butadienyl, pentadienyl, hexadienyl, heptadienyl, octadienyl, nonadienyl, decadienyl etc. without any further definition are meant unsaturated hydrocarbon groups with the corresponding number of carbon atoms and two double bonds, all the isomeric forms, i.e. (Z)/(E) isomers, being included where applicable.

By the terms propynyl, butynyl, pentynyl, hexynyl, heptynyl, octynyl, nonynyl, decynyl etc. without any further definition are meant unsaturated hydrocarbon groups with the corresponding number of carbon atoms and a triple bond, all the isomeric forms being included.

By the term heteroalkyl are meant groups which can be derived from the alkyl as defined above in its broadest sense if, in the hydrocarbon chains, one or more of the groups —CH₃ are replaced independently of one another by the groups —OH, —SH or —NH₂, one or more of the groups —CH₂— are replaced independently of one another by the groups —O—, —S— or —NH—, one or more of the groups

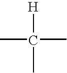

are replaced by the group

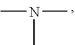

one or more of the groups ═CH— are replaced by the group ═N—, one or more of the groups ═CH₂ are replaced by the group ═NH or one or more of the groups —CH are replaced by the group ≡N, while overall there may only be a maximum of three heteroatoms in a heteroalkyl, there must be at least one carbon atom between two oxygen atoms and between two sulphur atoms or between one oxygen and one sulphur atom and the group as a whole must be chemically stable.

It is immediately apparent from the indirect definition/derivation from alkyl that heteroalkyl is made up of the sub-groups saturated hydrocarbon chains with heteroatom(s), heteroalkenyl and heteroalkynyl, and one further subdivision may be carried out into straight-chain (unbranched) and branched. If a heteroalkyl is substituted, the substitution may be mono- or polysubstitution in each case, at all the hydrogen-carrying oxygen, sulphur, nitrogen and/or carbon atoms, independently of one another. Heteroalkyl itself may be linked to the molecule as a substituent both via a carbon atom and via a heteroatom.

Typical examples are listed below:

dimethylaminomethyl; dimethylaminoethyl (1-dimethylaminoethyl; 2-dimethyl-aminoethyl); dimethylaminopropyl (1-dimethylaminopropyl, 2-dimethylaminopropyl, 3-dimethylaminopropyl); diethylaminomethyl; diethylaminoethyl (1-diethylaminoethyl, 2-diethylaminoethyl); diethylaminopropyl (1-diethylaminopropyl, 2-diethylamino-propyl, 3-diethylaminopropyl); diisopropylaminoethyl (1-diisopropylaminoethyl, 2-di-isopropylaminoethyl); bis-2-methoxyethylamino; [2-(dimethylamino-ethyl)-ethyl-amino]-methyl; 3-[2-(dimethylamino-ethyl)-ethyl-amino]-propyl; hydroxymethyl; 2-hydroxy-ethyl; 3-hydroxypropyl; methoxy; ethoxy; propoxy; methoxymethyl; 2-methoxyethyl etc.

Haloalkyl is derived from alkyl as hereinbefore defined in its broadest sense, when one or more hydrogen atoms of the hydrocarbon chain are replaced independently of one another by halogen atoms, which may be identical or different. It is immediately apparent from the indirect definition/derivation from alkyl that haloalkyl is made up of the sub-groups saturated halohydrocarbon chains, haloalkenyl and haloalkynyl, and further subdivision may be made into straight-chain (unbranched) and branched. If a haloalkyl is substituted, the substitution may be mono- or polysubstitution in each case, at all the hydrogen-carrying carbon atoms, independently of one another.

Typical examples are listed below:

—CF₃; —CHF₂; —CH₂F; —CF₂CF₃; —CHFCF₃; —CH₂CF₃; —CF₂CH₃; —CHFCH₃; —CF₂CF₂CF₃; —CF₂CH₂CH₃; —CF═CF₂; —CCl═CH₂; —CBr═CH₂; —CI═CH₂; —C≡C—CF₃; —CHFCH₂CH₃; —CHFCH₂CF₃ etc.

Halogen denotes fluorine, chlorine, bromine and/or iodine atoms.

Cycloalkyl is made up of the sub-groups monocyclic hydrocarbon rings, bicyclic hydro-carbon rings and spirohydrocarbon rings, while each sub-group may be further subdivided into saturated and unsaturated (cycloalkenyl). The term unsaturated means that in the ring system in question there is at least one double bond, but no aromatic system is formed. In bicyclic hydrocarbon rings two rings are linked such that they have at least two carbon atoms in common. In spirohydrocarbon rings one carbon atom (spiroatom) is shared by two rings. If a cycloalkyl is substituted, the substitution may be mono- or poly-substitution in each case, at all the hydrogen-carrying carbon atoms, independently of one another. Cycloalkyl itself may be linked to the molecule as substituent via any suitable position of the ring system.

Typical examples of individual sub-groups are listed below.

### Monocyclic Hydrocarbon Rings, Saturated:

cyclopropyl; cyclobutyl; cyclopentyl; cyclohexyl; cycloheptyl etc.

### Monocyclic Hydrocarbon Rings Unsaturated:

cycloprop-1-enyl; cycloprop-2-enyl; cyclobut-1-enyl; cyclobut-2-enyl; cyclopent-1-enyl; cyclopent-2-enyl; cyclopent-3-enyl; cyclohex-1-enyl; cyclohex-2-enyl; cyclohex-3-enyl; cyclohept-1-enyl; cyclohept-2-enyl; cyclohept-3-enyl; cyclohept-4-enyl; cyclobuta-1,3-dienyl; cyclopenta-1,4-dienyl; cyclopenta-1,3-dienyl; cyclopenta-2,4-dienyl; cyclohexa-1,3-dienyl; cyclohexa-1,5-dienyl; cyclohexa-2,4-dienyl; cyclohexa-1,4-dienyl; cyclohexa-2,5-dienyl etc.

### Bicyclic Hydrocarbon Rings (Saturated and Unsaturated):

bicyclo[2.2.0]hexyl; bicyclo[3.2.0]heptyl; bicyclo[3.2.1]octyl; bicyclo[2.2.2]octyl; bicyclo[4.3.0]nonyl(octahydroindenyl); bicyclo[4.4.0]decyl(decahydronaphthalene); bicycdo[2,2,1]heptyl(norbornyl); (bicyclo[2.2.1]hepta-2,5-dienyl(norborna-2,5-dienyl); bicyclo[2,2,1]hept-2-enyl(norbornenyl); bicyclo[4.1.0]heptyl(norcaranyl); bicyclo-[3.1.1]heptyl(pinanyl) etc.

### Spirohydrocarbon Rings (Saturated and Unsaturated):

spiro[2.5]octyl, spiro[3.3]heptyl, spiro[4.5]dec-2-enyl etc.

Cycloalkylalkyl denotes the combination of the above-defined groups alkyl and cycloalkyl, in each case in their broadest sense. The alkyl group as substituent is directly linked to the molecule and is in turn substituted by a cycloalkyl group. The alkyl and cycloalkyl may be linked in both groups via any carbon atoms suitable for this purpose. The respective sub-groups of alkyl and cycloalkyl are also included in the combination of the two groups.

Aryl denotes mono-, bi- or tricyclic carbon rings with at least one aromatic ring. If an aryl is substituted, the substitution may be mono- or polysubstitution in each case, at all the hydrogen-carrying carbon atoms, independently of one another. Aryl itself may be linked to the molecule as substituent via any suitable position of the ring system. Typical examples are listed below.

phenyl; naphthyl; indanyl (2,3-dihydroindenyl); 1,2,3,4-tetrahydronaphthyl; fluorenyl etc.

Arylalkyl denotes the combination of the groups alkyl and aryl as hereinbefore defined, in each case in their broadest sense. The alkyl group as substituent is directly linked to the molecule and is in turn substituted by an aryl group. The alkyl and aryl may be linked in both groups via any carbon atoms suitable for this purpose. The respective sub-groups of alkyl and aryl are also included in the combination of the two groups.

Typical examples are listed below:

benzyl; 1-phenylethyl; 2-phenylethyl; phenylvinyl; phenylallyl etc.

Heteroaryl denotes monocyclic aromatic rings or polycyclic rings with at least one aromatic ring, which, compared with corresponding aryl or cycloalkyl, contain instead of one or more carbon atoms one or more identical or different heteroatoms, selected independently of one another from among nitrogen, sulphur and oxygen, while the resulting group must be chemically stable. If a heteroaryl is substituted, the substitution may be mono- or polysubstitution in each case, at all the hydrogen-carrying carbon and/or nitrogen atoms, independently of one another. Heteroaryl itself as substituent may be linked to the molecule via any suitable position of the ring system, both carbon and nitrogen. Typical examples are listed below.

### Monocyclic Heteroaryls:

furyl; thienyl; pyrrolyl; oxazolyl; thiazolyl; isoxazolyl; isothiazolyl; pyrazolyl; imidazolyl; triazolyl; tetrazolyl; oxadiazolyl; thiadiazolyl; pyridyl; pyrimidyl; pyridazinyl; pyrazinyl; triazinyl; pyridyl-N-oxide; pyrrolyl-N-oxide; pyrimidinyl-N-oxide; pyridazinyl-N-oxide; pyrazinyl-N-oxide; imidazolyl-N-oxide; isoxazolyl-N-oxide; oxazolyl-N-oxide; thiazolyl-N-oxide; oxadiazolyl-N-oxide; thiadiazolyl-N-oxide; triazolyl-N-oxide; tetrazolyl-N-oxide etc.

### Polycyclic Heteroaryls:

indolyl; isoindolyl; benzofuryl; benzothienyl; benzoxazolyl; benzothiazolyl; benzisoxazolyl; benzoisothiazolyl; benzimidazolyl; indazolyl; isoquinolinyl; quinolinyl; quinoxalinyl; cinnolinyl; phthalazinyl; quinazolinyl; benzotriazinyl; indolizinyl; oxazolopyridyl; imidazopyridyl; naphthyridinyl; indolinyl; isochromanyl; chromanyl; tetrahydroisoquinolinyl; isoindolinyl; isobenzotetrahydrofuryl; isobenzotetrahydrothienyl; isobenzothienyl; benzoxazolyl; pyridopyridyl; benzotetrahydrofuryl; benzotetrahydro-thienyl; purinyl; benzodioxolyl; phenoxazinyl; phenothiazinyl; pteridinyl; benzothiazolyl; imidazopyridyl; imidazothiazolyl; dihydrobenzisoxazinyl; benzisoxazinyl; benzoxazinyl; dihydrobenzisothiazinyl; benzopyranyl; benzothiopyranyl; cumarinyl; isocumarinyl; chromonyl; chromanonyl; tetrahydroquinolinyl; dihydroquinolinyl; dihydroquinolinonyl; dihydroisoquinolinonyl; dihydrocumarinyl; dihydroisocumarinyl; isoindolinonyl; benzodioxanyl; benzoxazolinonyl; quinolinyl-N-oxide; indolyl-N-oxide; indolinyl-N-oxide; isoquinolyl-N-oxide; quinazolinyl-N-oxide; quinoxalinyl-N-oxide; phthalazinyl-N-oxide; indolizinyl-N-oxide; indazolyl-N-oxide; benzothiazolyl-N-oxide; benzimidazolyl-N-oxide; benzothiopyranyl-5-oxide and benzothiopyranyl-S,S-dioxide etc.

Heteroarylalkyl denotes the combination of the alkyl and heteroaryl groups defined hereinbefore, in each case in their broadest sense. The alkyl group as substituent is directly linked to the molecule and is in turn substituted by a heteroaryl group. The linking of the alkyl and heteroaryl may be achieved on the alkyl side via any carbon atoms suitable for this purpose and on the heteroaryl side by any carbon or nitrogen atoms suitable for this purpose. The respective sub-groups of alkyl and heteroaryl are also included in the combination of the two groups.

By the term heterocycloalkyl are meant groups which are derived from the cycloalkyl as hereinbefore defined if in the hydrocarbon rings one or more of the groups —CH₂— are replaced independently of one another by the groups —O—, —S— or —NH— or one or more of the groups ═CH— are replaced by the group ═N—, while not more than five heteroatoms may be present in total, there must be at least one carbon atom between two oxygen atoms and between two sulphur atoms or between one oxygen and one sulphur atom and the group as a whole must be chemically stable. Heteroatoms may simultaneously be present in all the possible oxidation stages (sulphur→sulphoxide —SO—, sulphone —SO₂—; nitrogen→N-oxide). It is immediately apparent from the indirect definition/derivation from cycloalkyl that heterocycloalkyl is made up of the sub-groups monocyclic hetero-rings, bicyclic hetero-rings and spirohetero-rings, while each sub-group can also be further subdivided into saturated and unsaturated (heterocycloalkenyl). The term unsaturated means that in the ring system in question there is at least one double bond, but no aromatic system is formed. In bicyclic hetero-rings two rings are linked such that they have at least two atoms in common. In spirohetero-rings one carbon atom (spiroatom) is shared by two rings. If a heterocycloalkyl is substituted, the substitution may be mono- or polysubstitution in each case, at all the hydrogen-carrying carbon and/or nitrogen atoms, independently of one another. Heterocycloalkyl itself as substituent may be linked to the molecule via any suitable position of the ring system.

Typical examples of individual sub-groups are listed below.

### Monocyclic Heterorings (Saturated and Unsaturated):

tetrahydrofuryl; pyrrolidinyl; pyrrolinyl; imidazolidinyl; thiazolidinyl; imidazolinyl; pyrazolidinyl; pyrazolinyl; piperidinyl; piperazinyl; oxiranyl; aziridinyl; azetidinyl; 1,4-dioxanyl; azepanyl; diazepanyl; morpholinyl; thiomorpholinyl; homomorpholinyl; homopiperidinyl; homopiperazinyl; homothiomorpholinyl; thiomorpholinyl-5-oxide; thiomorpholinyl-S,S-dioxide; 1,3-dioxolanyl; tetrahydropyranyl; tetrahydrothiopyranyl; [1,4]-oxazepanyl; tetrahydrothienyl; homothiomorpholinyl-S,S-dioxide; oxazolidinonyl; dihydropyrazolyl; dihydropyrrolyl; dihydropyrazinyl; dihydropyridyl; dihydropyrimidinyl; dihydrofuryl; dihydropyranyl; tetrahydrothienyl-5-oxide; tetrahydrothienyl-S,S-dioxide; homothiomorpholinyl-5-oxide; 2,3-dihydroazet; 2H-pyrrolyl; 4H-pyranyl; 1,4-dihydropyridinyl etc.

### Bicyclic Heterorings (Saturated and Unsaturated):

8-azabicyclo[3.2.1]octyl; 8-azabicyclo[5.1.0]octyl; 2-oxa-5-azabicyclo[2.2.1]heptyl; 8-oxa-3-aza-bicyclo[3.2.1]octyl; 3.8-diaza-bicyclo[3.2.1]octyl; 2.5-diaza-bicyclo-[2.2.1]heptyl; 1-aza-bicyclo[2.2.2]octyl; 3.8-diaza-bicyclo[3.2.1]octyl; 3.9-diaza-bicyclo[4.2.1]nonyl; 2.6-diaza-bicyclo[3.2.2]nonyl etc.

### Spiro-Heterorings (Saturated and Unsaturated):

1,4-dioxa-spiro[4.5]decyl; 1-oxa-3.8-diaza-spiro[4.5]decyl; and 2,6-diaza-spiro[3.3]heptyl; 2,7-diaza-spiro[4.4]nonyl; 2,6-diaza-spiro[3.4]octyl; 3,9-diaza-spiro[5.5]undecyl; 2,8-diaza-spiro[4.5]decyl etc.

Heterocycloalkylalkyl denotes the combination of the alkyl and heterocycloalkyl groups defined hereinbefore, in each case in their broadest sense. The alkyl group as substituent is directly linked to the molecule and is in turn substituted by a heterocycloalkyl group. The linking of the alkyl and heterocycloalkyl may be achieved on the alkyl side via any carbon atoms suitable for this purpose and on the heterocycloalkyl side by any carbon or nitrogen atoms suitable for this purpose. The respective sub-groups of alkyl and heterocycloalkyl are also included in the combination of the two groups.

The term “substituted” indicates that a hydrogen atom which is bound directly to the atom in question is replaced by another atom or another group of atoms. Bivalent substituents such as for example ═O, ═S, ═NR, ═NOR, ═NNRR, ═NN(R)C(O)NRR, ═N₂ or the like can only be substituents at carbon atoms. They require exchanging for two geminal hydrogen atoms, i.e. hydrogen atoms which are bound to the same carbon atom saturated before the substitution. Substitution by a bivalent substituent is therefore only possible at the groups —CH₃ and —CH₂—, not at the groups

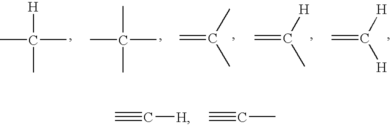

and not at aromatic carbon atoms.

Additionally, by the term “suitable substituent/suitable group” is meant a substituent which on the one hand is suitable on account of its valency and on the other hand leads to a system with chemical stability.

List of abbreviations

| | |
|---|---|
| 9-BBN | 9-borabicyclo[3.3.1]nonane |
| Ac | acetyl |
| Bn | benzyl |
| Boc | tert.-butyloxycarbonyl |
| Bu | butyl |
| c | concentration |
| chex | cyclohexane |
| dba | dibenzylideneacetone |
| DBAD | di-tert.-butyl-azodicarboxylate |
| TLC, TLC | thin layer chromatography |
| DCM | dichloromethane |
| DEA | diethylamine |
| DIPEA | N-ethyl-N,N-diisopropylamine |
| | (Hünig base) |
| DMA | N,N-dimethylacetamide |
| DMAP | 4-N,N-dimethyl-aminopyridine |
| DME | 1,2-dimethoxyethane |
| DMF | N,N-dimethylformamide |
| DMSO | dimethylsulphoxide |
| EA | ethyl acetate (ethyl acetate) |
| eq | equivalent(s) |
| ESI | electron spray ionization |
| Et | ethyl |
| h | hour |
| hex | hexyl |
| HPLC | high performance liquid chromatography |
| Hünig base | N-ethyl-N,N-diisopropylamine |
| i | iso |
| IR | infrared spectroscopy |
| cat., cat | catalyst, catalytic |
| conc. | concentrated |
| bp., b.p. | boiling point |
| LC | liquid chromatography |
| LHMDS | lithium hexamethyl disilazane |
| soln. | solution |
| Me | methyl |
| min | minutes |
| MPLC | medium pressure liquid chromatography |
| MS | mass spectrometry |
| NMP | N-methylpyrrolidone |
| n.a. | not available |
| Ph | phenyl |
| Pr | propyl |
| PS | polystyrene |
| Py | pyridine |
| rac | racemic |
| R_{f} (Rf) | retention factor |
| RP | reversed phase |
| RT | ambient temperature |
| TBTU | O-(benzotriazol-1-yl)-N,N,N′,N′-tetramethyluronium |
| | tetrafluoroborate |
| temp. | temperature |
| tert. | tertiary |
| Tf | triflate |
| Tfa | trifluoroacetic acid |
| THF | tetrahydrofuran |
| TsOH | para-toluenesulphonic acid |
| UV | ultraviolet |

Features and advantages of the present invention will become apparent from the following detailed Examples, which illustrate the basics of the invention by way of example, without limiting its scope.

### Preparation of the Compounds According to the Invention

### General

All the reactions are carried out—unless stated otherwise—in commercially obtainable apparatus using methods conventionally used in chemical laboratories.

Air- and/or moisture-sensitive starting materials are stored under protective gas and corresponding reactions and manipulations using them are carried out under protective gas (nitrogen or argon).

Microwave reactions are carried out in an EMRY OPTIMIZER made by Personal Chemistry in sealed containers (5 or 20 mL), preferably with stirring.

### Chromatography

For the preparative medium pressure chromatography (MPLC, normal phase) silica gel is used which is made by Millipore (named: Granula Silica Si-60A 35-70 μm) or C-18 RP-silica gel (RP-phase) made by Macherey Nagel (named: Polygoprep 100-50 C18). The thin layer chromatography is carried out on ready-made silica gel 60 TLC plates on glass (with fluorescence indicator F-254) made by Merck.

For the preparative high pressure chromatography (HPLC) columns made by Waters (named: XTerra Prep. MS C18, 5 μM, 30×100 mm or XTerra Prep. MS C18, 5 μm, 50×100 mm OBD or Symmetrie C18, 5 μm, 19×100 mm) are used, the analytical HPLC (reaction control) is carried out with columns made by Agilent (named: Zorbax SB-C8, 5 μm, 21.2×50 mm).

For the chiral high pressure chromatography (HPLC) columns made by Daicel Chemical Industries, Ltd. are used (named: Chiralpak AD-H or Chiralpak AS or Chiracel OD-RH or Chiracel OD-H or Chiracel OJ-H in various sizes and 5 μm material).

### HPLC Mass Spectroscopy/UV Spectrometry

The retention times/MS-ESI⁺ for characterising the examples are obtained using an HPLC-MS apparatus (high performance liquid chromatography with mass detector) made by Agilent. Compounds that elute with the injection peak are given the retention time t_{Ret.}=0.00.

The apparatus has the following specification:

| | |
|---|---|
| Column: | Waters, Xterra MS C18, 2.5 μm, 2.1 × 30 mm, |
| | Part. No. 186000592 |
| Eluant: | A: H₂O with 0.1% HCOOH; B: acetonitrile |
| | (HPLC grade) |
| Detection: | MS: Positive and negative mode |
| Mass range: | 120-900 m/z |
| Fragmentor: | 120 |
| EMV Gain: | 1; Threshold: 150; Stepsize: 0.25; UV: 254 nm; |
| | Bandwide: 1 |
| Injection: | Inj. Vol. 5 μL |
| Separation: | Flow 1.10 mL/min |
| Column temp.: | 40° C. |
| Gradient: | 0.00 min: 5% solvent B |
| | 0.00-2.50 min: 5% → 95% solvent B |
| | 2.50-2.80 min: 95% solvent B |
| | 2.81-3.10 min: 95% → 5% solvent B |

The compounds according to the invention may be prepared by the methods of synthesis described below, in which the substituents of the general formulae have the meanings specified hereinbefore. These methods are intended to illustrate the invention without restricting it to their content or limiting the scope of the compounds claimed to these Examples. Where the preparation of the starting compounds is not described, they are commercially obtainable or may be prepared analogously to known compounds or methods described herein. Substances described in the literature are prepared according to the published methods of synthesis.

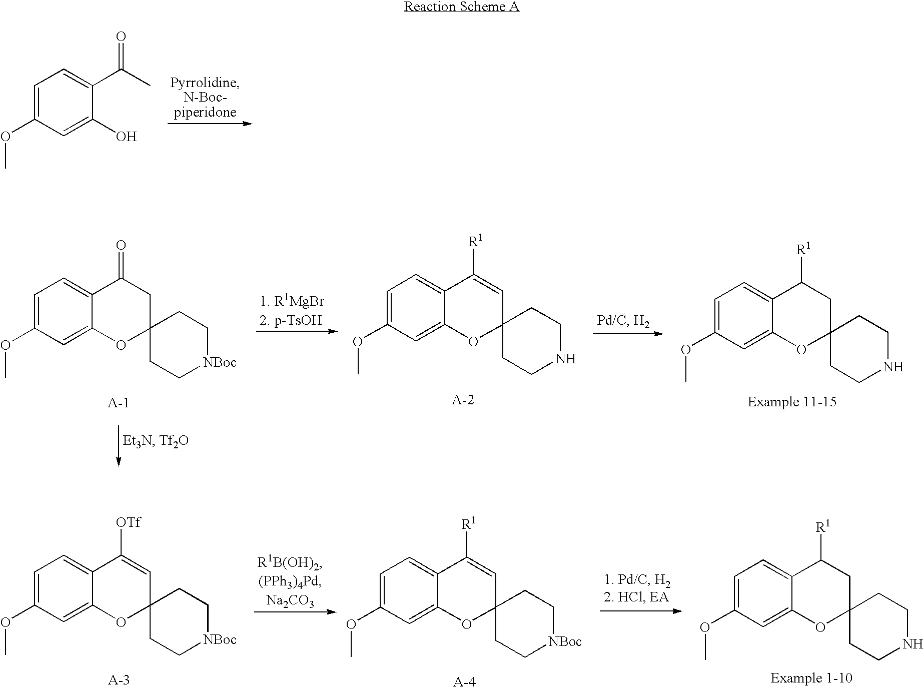

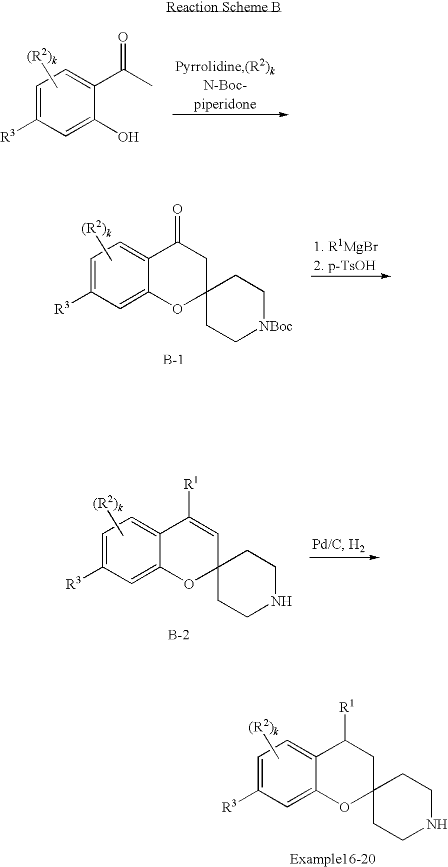

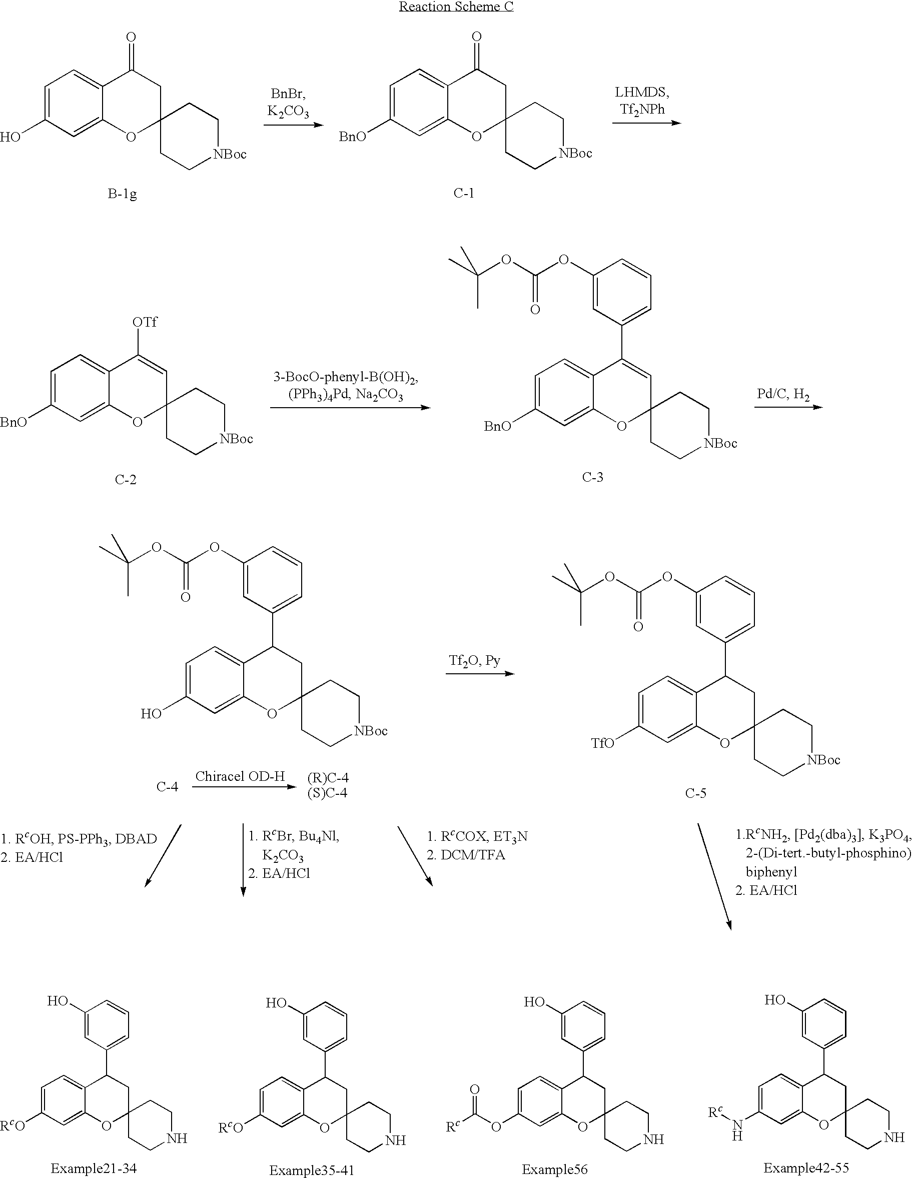

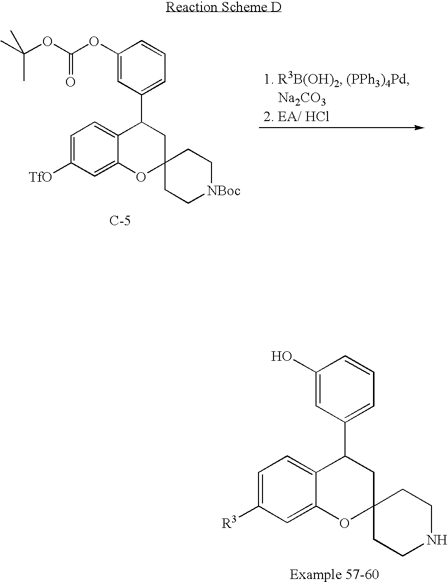

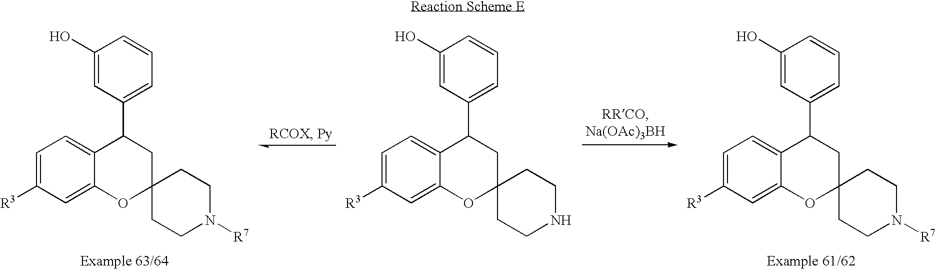

### Synthesis of the Starting Compounds

### Synthesis of A-1

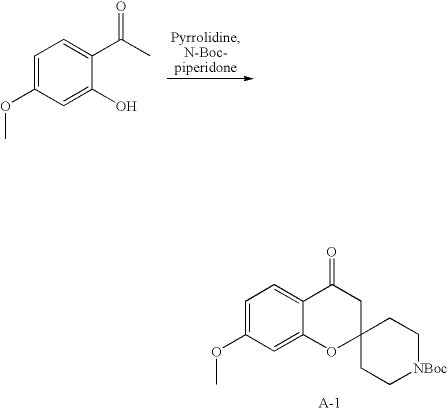

2-hydroxy-4-methoxyacetophenone (5.12 g, 30.5 mmol) is dissolved in MeOH under a protective gas atmosphere and combined with pyrrolidine. After 30 min stirring at RT tert.-butyl 4-piperidone-N-carboxylate (6.2 g, 30.51 mmol) is added and the mixture is refluxed for 68 h. After cooling to RT the solvents are eliminated in vacuo. The residue is taken up in EA, and extracted with aqueous 1 N HCl and aqueous saturated NaHCO₃ soln. After drying the organic phase on Na₂SO₄, filtering and eliminating the solvent in vacuo A-1 is obtained.

TLC: R_{f}=0.61 (chex:EA=1:1); LC-MS: t_{Ret.}=2.08 min

### General Method of Synthesising Compounds of Type A-2:

A-1 is dissolved in THF, combined with a Grignard solution (in diethyl ether or THF) and refluxed. Once the end of the reaction is confirmed by HPLC-MS, the mixture is cooled to RT, poured onto aqueous saturated NH₄Cl soln. and extracted 3× with EA. The combined organic extracts are dried on Na₂SO₄, filtered and evaporated down. The residue is taken up in p-TsOH in toluene and refluxed for 16 h. After cooling to RT the mixture is poured onto aqueous NaHCO₃ soln., the organic phase is separated off and dried on Na₂SO₄, filtered and evaporated down. The residue is purified by column chromatography.

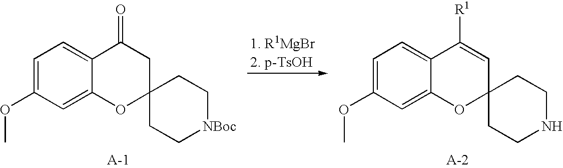

| # | R¹ | t_{Ret.} (HPLC) [min] | MS (ESI+) [M + H]⁺ |
|---|---|---|---|
| A-2a | 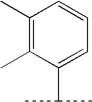 | 1.73 | 336 |
| A-2b | 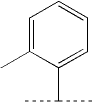 | n.a. | 322 |
| A-2c | 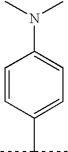 | 1.40 | 351 |
| A-2d | 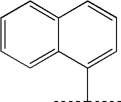 | 1.70 | 358 |
| A-2e | 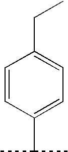 | 1.75 | 336 |
| A-2f | 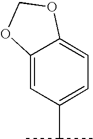 | 1.56 | 352 |
| A2g | 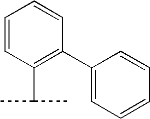 | 1.75 | 384 |

### Synthesis of A-3:

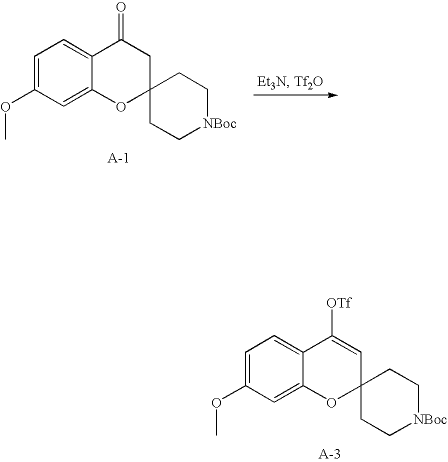

A-1 (102 mg, 0.294 mmol) is dissolved in dry DCM under a protective gas atmosphere, combined with Et₃N (90 μL, 0.646 mmol) and the mixture is cooled in a bath of dry ice/acetone. Trifluoromethanesulphonic anhydride (109 μL, 0.646 mmol) is added and the mixture is stirred for 1 h in the ice bath and for another 30 min at RT. The reaction mixture is combined with chex, filtered through Celite, washed with EA and evaporated down. After purification by silica gel chromatography A-3 is obtained.

TLC: R_{f}=0.86 (chex:EA=1:1)

### General Method of Synthesising Compounds of Type A-4:

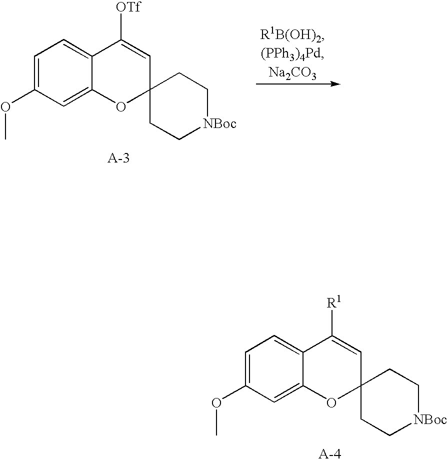

A-3 (1 mmol), boric acid (1.5 eq), Na₂CO₃ (2.0 eq) and tetrakis(triphenylphosphine)palladium(0) are suspended in dioxane/water (7 mL, 5:2) and stirred for 10 min at 150° C. in an argon atmosphere in the microwave reactor. The cooled reaction mixture is divided between saturated NH₄Cl soln. and EA, the organic phase is washed 1× with 1 N HCl, saturated NaHCO₃ soln. and saturated NaCl soln., dried on Na₂SO₄, filtered and the solvent is eliminated in vacuo. After silica gel chromatography (eluant: chex/EA, gradient 2%-50% EA) the title compounds are obtained.

| # | R¹ | t_{Ret.} (HPLC) [min] | MS |
|---|---|---|---|
| A-4a | 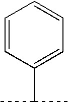 | 2.6 | 408 (M + H)⁺ |
| A-4b | 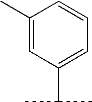 | 2.7 | 422 (M + H)⁺ |
| A-4c | 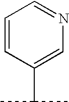 | 2.04 | 409 (M + H)⁺ |
| A-4d | 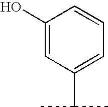 | 2.33 | 422 (M − H)⁻ |
| A-4e | 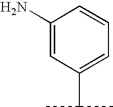 | n.a. | 367 (M + H)⁺ |
| A-4f | 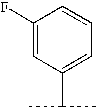 | n.a. | 448 (M + Na)⁺ |
| A-4g | 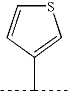 | n.a. | 358 (M + H)⁺ |
| A-4h | 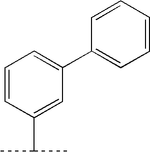 | 3.06 | 484 (M + H)⁺ |
| A-4i | 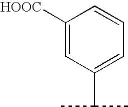 | 2.47 | 450 (M − H)⁻ |

### General Method of Synthesising Compounds of Type B-1:

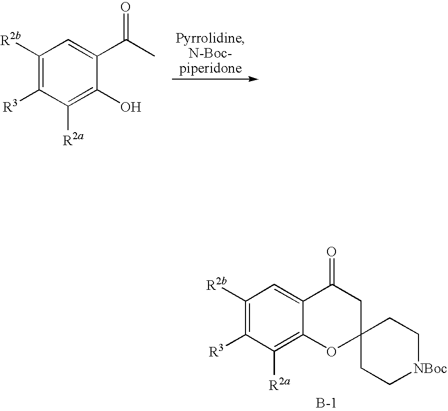

Under an argon atmosphere the substituted acetophenone (25 mmol) is dissolved in MeOH (40 mL) and combined with pyrrolidine (1 eq). After 20 min stirring at RT tert.butyl 4-piperidone-N-carboxylate (1 eq) is added and then the mixture is refluxed for 62 h. The solvents are eliminated in vacuo and the residue is taken up in EA. After washing the organic phase with 1 N HCl and saturated NaCl soln. the mixture is dried on Na₂SO₄, filtered and the title compounds are purified by silica gel chromatography (eluant: chex/EA, gradient 5%-75% EA).

| # | R^{2a} | R³ | R^{2b} | t_{Ret.} (HPLC) [min] | MS |
|---|---|---|---|---|---|
| B-1a | H | CH₃ | H | 2.19 | n.a. |
| B-1b | 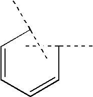 | | H | 2.29 | 312 (M + H)⁺-tert.-butyl |
| B-1c | OH | OH | H | 1.67 | 348 (M − H)⁻ |
| B-1d | H | OCH₂CH₃ | H | 2.20 | 262 (M + H)⁺ |
| B-1e | H | OCH₃ | Cl | 2.20 | 404 (M + Na)⁺ |
| B-1f | CH₃ | OH | H | 1.94 | 346 (M − H)⁻ |
| B-1g | H | OH | H | 1.83 | 332 (M − H)⁻ |
| B-1h | H | CN | H | n.a. | 341 (M − H)⁻ |
| B-1i | H | 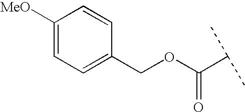 | H | n.a. | 382 (M + H)⁺-tert.-butyl |
| B-1j | H | Br | H | 2.31 | 340/342 (M + H)⁺-tert.-butyl |

### General Method of Synthesising Compounds of Type B-2:

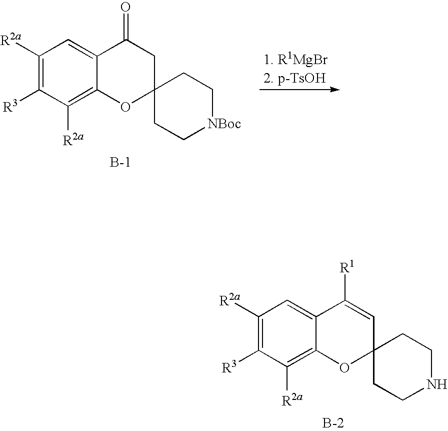

B-1 (0.4 mmol) is dissolved in THF (3 mL), combined with Grignard reagent (2 eq) in diethyl ether or THF and refluxed for 16 h. After cooling to RT the mixture is poured onto saturated aqueous NH₄Cl soln. and extracted 3× with EA. The combined organic extracts are dried on Na₂SO₄, filtered and evaporated down. The crude product is refluxed for 16 h with catalytic amounts of p-TsOH in toluene. The solvents are evaporated off in vacuo and the residue is divided between saturated NaHCO₃ soln. and EA. The organic phases are dried on Na₂SO₄, filtered, evaporated down and the crude product is chromatographed by preparative HPLC (RP-18).

| | | | | | t_{Ret} (HPLC) | MS |
|---|---|---|---|---|---|---|
| # | R¹ | R^{2a} | R³ | R^{2b} | [min] | (M + H)⁺ |
| B-2a | 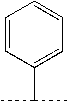 | H | CH₃ | H | 1.64 | 292 |
| B-2b | 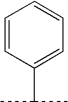 | 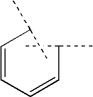 | | H | 1.72 | 328 |
| B-2c | 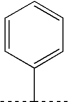 | 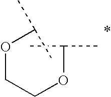 | | H | 1.56 | 336 |
| B-2d | 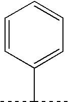 | H | OCH₂CH₃ | H | 1.67 | 322 |
| B-2e | 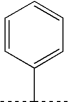 | H | OCH₃ | Cl | 1.68 | 342 |
| B-2f | 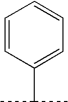 | CH₃ | OCH₃** | H | 1.64 | 322 |

*The educt for B-2c is prepared by reacting B-1c with 1,2-dibromoethane (2.6 eq) and K₂CO₃ (8.6 eq) in NMP at RT in the ultrasound reactor (Branson Sonifier) and purified by silica gel chromatography.
**The educt for B-2f is prepared by alkylation of B-1f with dimethylsulphate/K₂CO₃ in acetone and used in the reaction without any further purification.

### Synthesis of C-1:

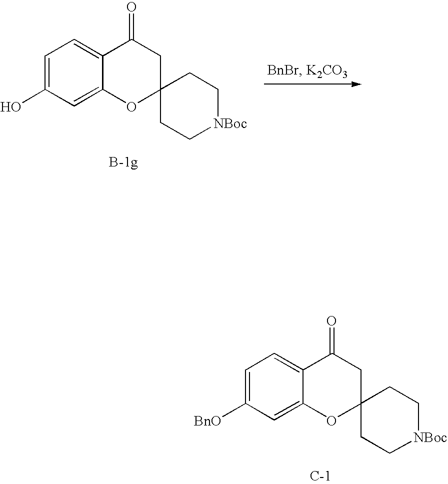

B-1 g (11.7 g, 35.1 mmol) and K₂CO₃ (21.1 g, 151.1 mmol) are suspended in acetone (100 mL), combined with benzylbromide and refluxed for 62 h. The precipitate is filtered off, washed with EA and the combined organic phases are evaporated to dryness in vacuo. The residue is purified by silica gel chromatography (eluant: chex/EA, gradient 2%-100% EA). (M−H)-=422; LC-MS: t_{Ret.}=2.38 min.

### Synthesis of C-2:

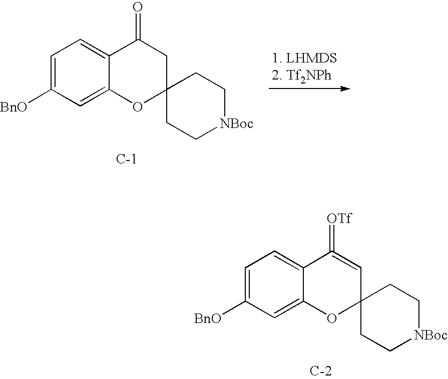

A solution of C-1 (6.9 g, 16.3 mmol) in THF (30 mL) is cooled to −78° C. under argon in a bath of acetone/dry ice, combined with a 1 M solution of LHMDS in THF (24.44 mL, 24.44 mmol) and stirred for 1 h at −78° C. to −60° C. Tf₂NPh (8.82 g, 24.44 mmol) is added in one go, the cooling bath is removed and the reaction mixture is stirred for 2 h at RT. Then the mixture is divided between saturated aqueous NH₄Cl soln and EA, the organic phase is dried on Na₂SO₄, filtered and evaporated down. The residue is dissolved in chex/EA (15:1) and chromatographed by Gilson-NP-MPLC (100 g silica gel cartridge, eluant: chex/EA, gradient 1%-50% EA). (M+H)⁺=500; LC-MS: t_{Ret.}=2.75 min.

### Synthesis of C-3:

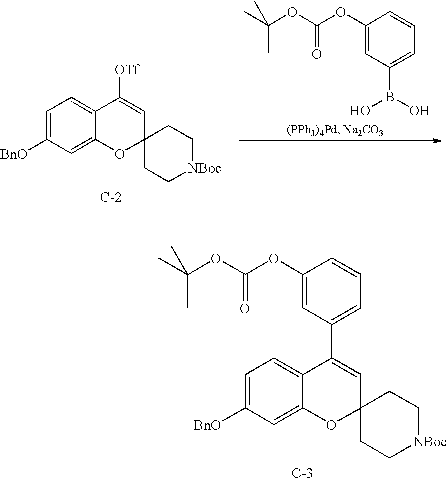

C-2 (8.63 g, 15.53 mmol), (3-tert.-butoxycarboxyphenyl) boric acid (4.15 g, 17.1 mmol), Na₂CO₃ (2.47 g, 23.3 mmol) and tetrakis(triphenylphosphine)palladium(0) (1.81 g, 1.55 mmol) are suspended in dioxane/water (28 mL, 5:2) under argon and then heated to 100° C. in the microwave (Anton Paar) for 10 min. After cooling to RT the mixture is divided between EA and saturated NaCl soln., the organic phase is dried on Na₂SO₄ and filtered through silica gel. After the solvent has been eliminated in vacuo the residue is purified by flash chromatography (1000 g silica gel, eluant chex/EA, gradient 100% chex-20% EA). TLC: R_{f}=0.45 (chex:EA 4:1); LC-MS: t_{Ret.}=2.92 min.

### Synthesis of C-4:

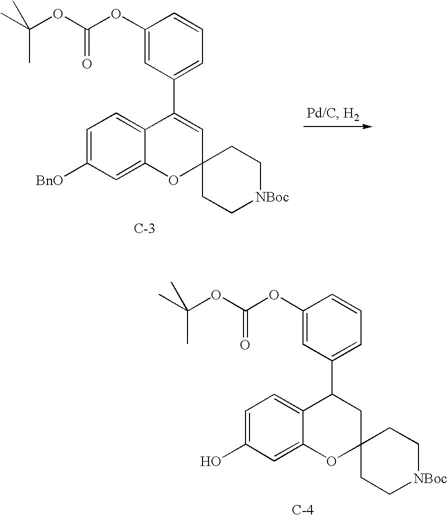

A solution of C-3 (1.01 g, 1.68 mmol) in THF (30 mL) is combined under argon with Pd/C (10% on activated charcoal, 355 mg). Argon is replaced by hydrogen and stirred for 24 h under 10 bar H₂ at RT. The reaction mixture is filtered through Celite, washed with EA and evaporated down. LC-MS: t_{Ret.}=2.45 min, (M-2Boc)⁺=400.

The racemic C-4 is separated on a Chiracel OD-H-column (5 μm, 250×4.6 mm). Eluant: n-heptane/ethanol/diethylamine (90/10/0.1) The two enantiomers elute at t_{Ret.}=7 min and at t_{Ret.}=10.7-11.0 min, respectively.

### Synthesis of C-5:

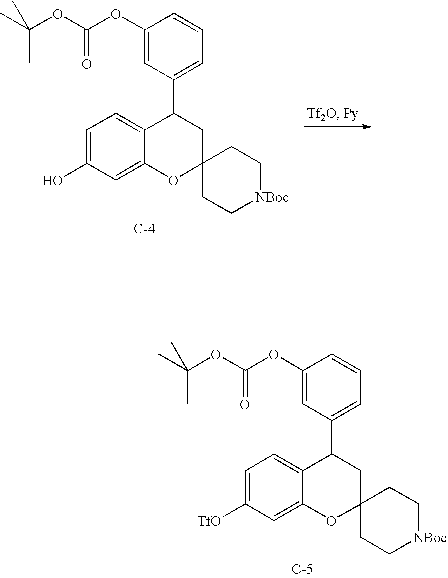

A solution of C-4 (2.0 g, 3.91 mmol) in DCM (10 mL) and pyridine (0.629 mL, 7.82 mmol) is combined with trifluoromethanesulphonic anhydride (0.790 mL, 4.69 mmol) under argon at 0° C. and stirred for 5 min at RT. The reaction mixture is divided between DCM and 1 N HCl (10 mL) and the organic phase is washed with saturated NaHCO₃ soln., dried and evaporated down. The title compound is used in the next reaction without any further purification. LC-MS: t_{Ret.}=2.85 min, (M+H)⁺=532.

### Example 1

### Synthesis Scheme A

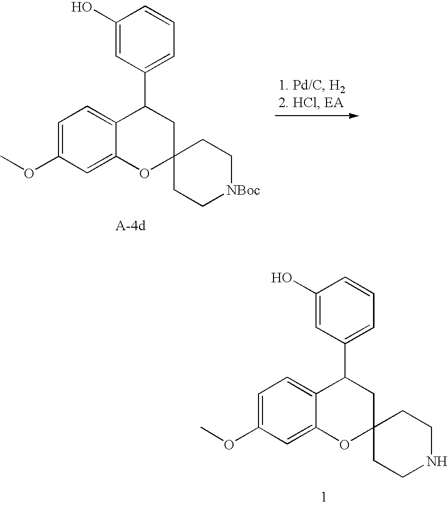

A solution of A-4-d (92 mg, 0.22 mmol) in MeOH/10% HCOOH (5 mL) is added drop-wise under argon to a suspension of Pd/C (182 mg, 10% on activated charcoal) in MeOH/10% HCOOH (1 mL) and the mixture is stirred for 16 h at RT. The reaction mixture is filtered through Celite, the filtrate is combined with 1 mL conc. HCl and stirred for 16 h at RT. The mixture is adjusted to pH 8 with saturated NaHCO₃ soln. and extracted with EA. The organic phase is dried on Na₂SO₄, filtered, evaporated down and the residue is dried under a high vacuum. The racemate is separated by chiral HPLC (Chiracel OD-H, eluant acetonitrile+0.1% diethylamine).

Examples 2-10 are prepared analogously (Table 1).

TABLE 1

| # | structure | t_{Ret.} (HPLC) [min] | MS (ESI+) [M + H]⁺ |
|---|---|---|---|
| 1 | 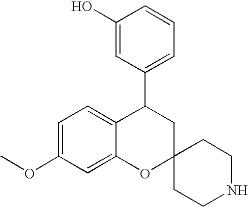 | 1.379 | 326 |
| 2 | 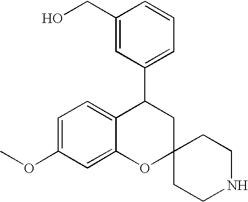 | 1.39 | 340 |
| 3 | 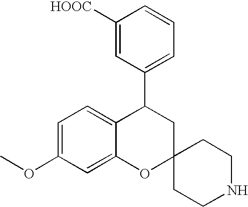 | 0.0 | 354 |
| 4 | 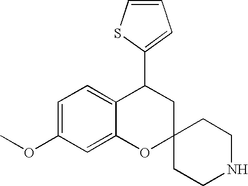 | 1.53 | 316 |
| 5 | 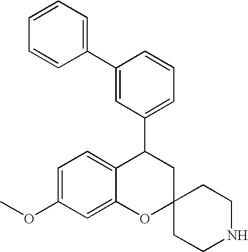 | 1.785 | 386 |
| 6 | 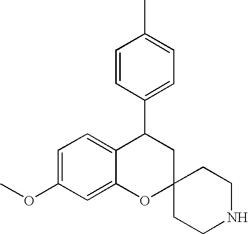 | 1.623 | 324 |
| 7 | 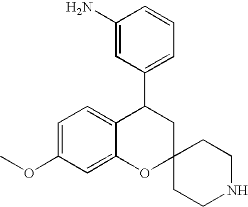 | 0.0 | 325 |
| 8 | 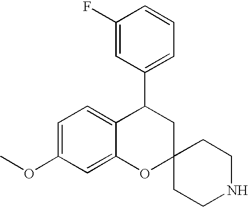 | 1.59 | 328 |
| 9 | 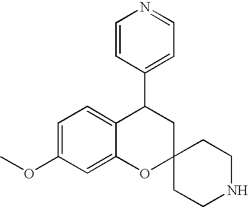 | 0.2 | 311 |
| 10 | 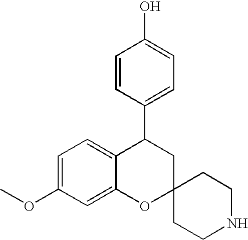 | 0.0 | 3.26 |

### Example 11

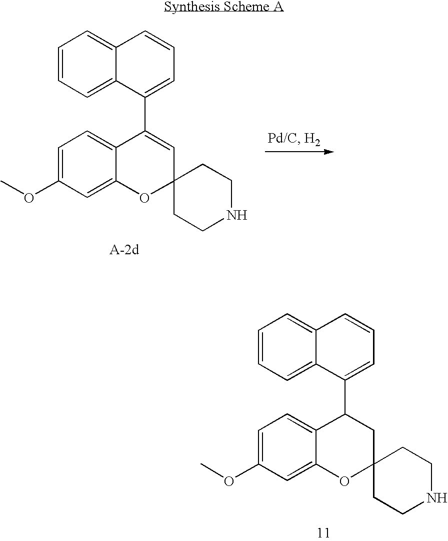

A solution of A-2d in MeOH/4% HCOOH (5 mL) is added dropwise to a suspension of Pd/C (10% on activated charcoal, 44.6 mg) in MeOH/4% HCOOH (3 mL) and the reaction mixture is stirred for 16 h at RT. The catalyst is eliminated by filtration through Celite and the filtrate is evaporated to dryness in vacuo. The residue is taken up in saturated Na—HCO₃ soln. and extracted 2× with EA. The combined organic extracts are dried on Na₂SO₄, filtered and evaporated down.

Examples 12-15 are prepared analogously (Table 2).

TABLE 2

| # | structure | t_{Ret.} (HPLC) [min] | MS (ESI+) [M + H]⁺ |
|---|---|---|---|
| 11 | 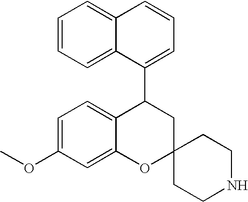 | 1.7 | 360 |
| 12 | 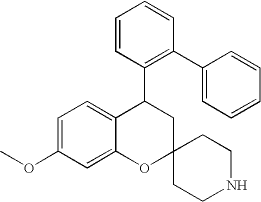 | 1.748 | 586 |
| 13 | 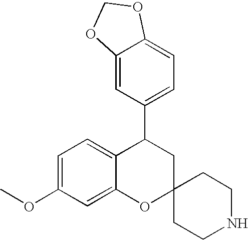 | 1.55 | 354 |
| 14 | 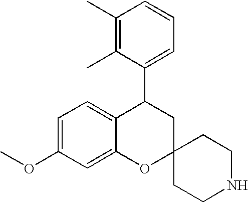 | 1.71 | 338 |
| 15 | 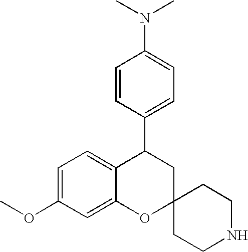 | 1.06 | 353 |

### Example 16

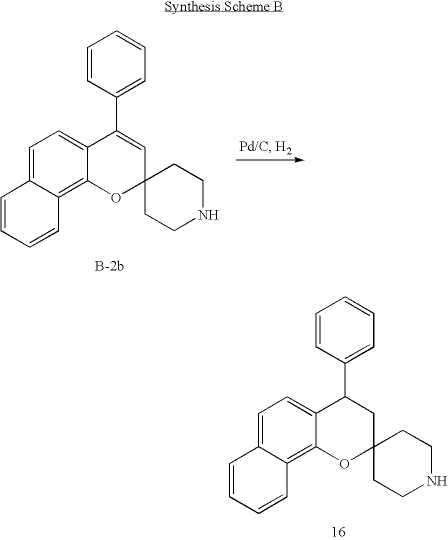

A solution of B-2b (99 mg, 0.30 mmol) is added dropwise under argon to a suspension of Pd/C (254 mg, 10% Pd on activated charcoal) in MeOH/2% HCOOH (3 mL) and the mixture is stirred overnight at RT. The catalyst is filtered off and the reaction mixture is evaporated down in vacuo. The residue is taken up in saturated NaHCO₃ soln., extracted 3× with EA, dried on Na₂SO₄, filtered and evaporated down. Some of the product obtained is separated off by chiral HPLC (Chiracel OD-H 150×2.1 mm; MeCN+0.1% DEA; 20° C.; 0.25 mL/min.).

Examples 17-20 are prepared analogously (Table 3).

TABLE 3

| # | structure | t_{Ret.} (HPLC) [min] | MS (ESI+) [M + H]⁺ |
|---|---|---|---|
| 16 | 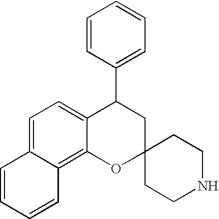 | 1.69 | 330 |
| 17 | 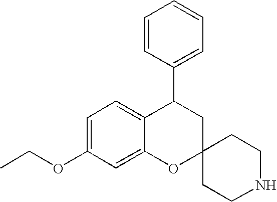 | 1.56 | 324 |
| 18 | 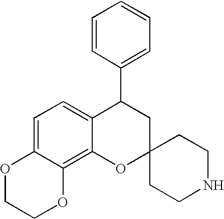 | 1.53 | 338 |
| 19 | 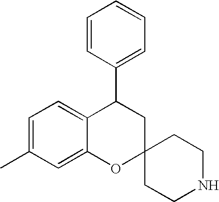 | 1.63 | 294 |
| 20 | 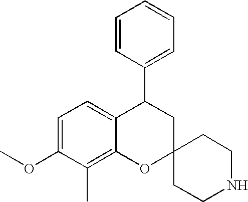 | 1.63 | 324 |

### Example 21

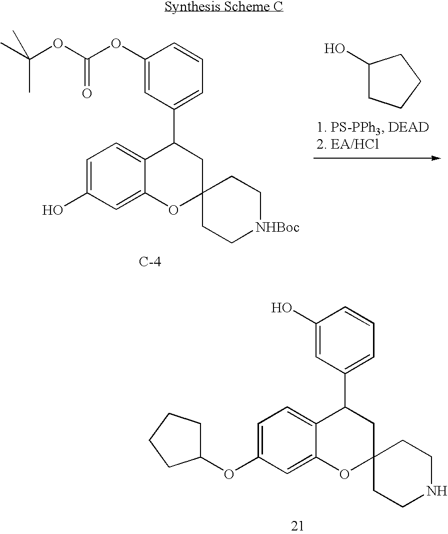

C-4 (157 mg, 0.31 mmol) and PS-triphenylphosphine (2.19 mmol/g, 617 mg, 1.35 mmol) are suspended in THF (5 mL) under an argon atmosphere and cyclopentanol (70 μL, 0.76 mmol) is added with stirring. Then the mixture is combined with a solution of di-tert.-butylazodicarboxylate (DBAD, 231 mg, 0.92 mmol) in THF (2 mL) and stirred for 30 min at RT. For working up the mixture is filtered, washed with DCM and EA and after the addition of a few drops of Et₃N it is chromatographed by Gilson-NP-MPLC (20 g isolute-HM-N silica gel cartridge, eluant: chex/EA, gradient 1%-50% EA). The residue (111 mg) is taken up in 10 mL EA, combined with 1 mL conc. HCl and stirred overnight at RT. Saturated NaHCO₃ soln. is added and the mixture is extracted with EA. The organic phases are dried on Na₂SO₄, filtered and evaporated down.

Examples 22-34 are prepared analogously (Table 4).

TABLE 4

| # | structure | t_{Ret.} (HPLC) [min] | MS (ESI+) [M + H]⁺ |
|---|---|---|---|
| 21 | 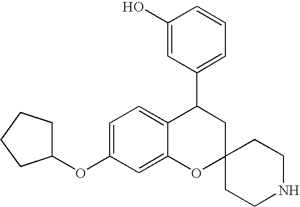 | 1.66 | 380 |
| 22 | 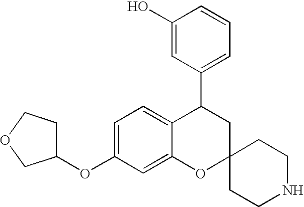 | n.a. | 382 |
| 23 | 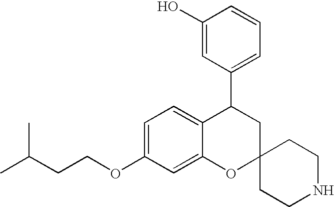 | n.a. | 382 |
| 24 | 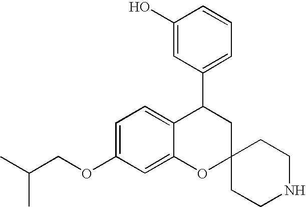 | 1.66 | 368 |
| 25 | 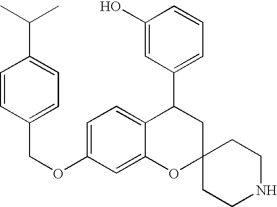 | 1.87 | 444 |
| 26 | 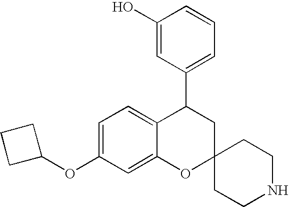 | 1.57 | 366 |
| 27 | 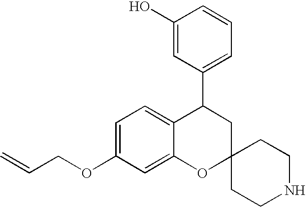 | 1.50 | 352 |
| 28 | 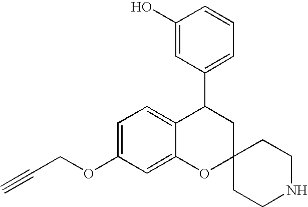 | 1.44 | 350 |
| 29 | 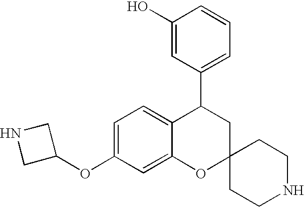 | 0.0 | 367 |
| 30 | 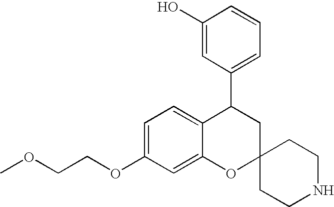 | 0.0 | 370 |
| 31* | 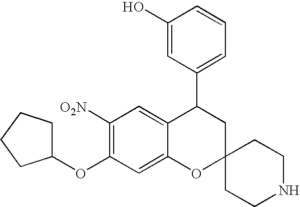 | 1.62 | 425 |
| 32** | 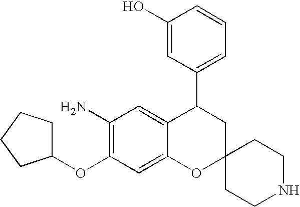 | 0.0 | 395 |
| 33 | 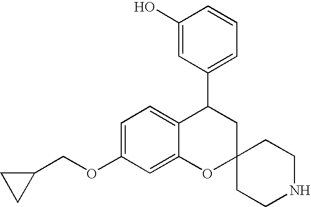 | 1.56 | 366 |
| 34*** | 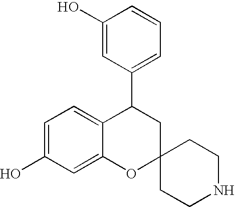 | 0.0 | 312 |

*To synthesise Example 31 first of all C-4 is nitrated with Fe(NO₃)₃ in dioxane at 40° C. in the 6-position, otherwise the reaction sequence runs analogously.
**Example 32 is prepared from Example 31 by hydrogenation with Pd/C in THF/MeOH (2:1).
***Example 34 is obtained directly from C-4 (12 mg, 23 μmol) by cleaving the Boc-protective groups in EA (10 mL) and conc. HCl (1 mL) without prior Mitsunobu reaction (16 h, RT).

### Example 35

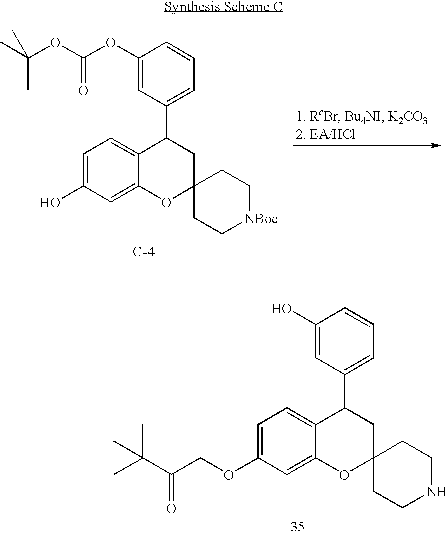

C-4 (70 mg, 0.37 mmol), K₂CO₃ (28 mg, 0.20 mmol), tetrabutylammonium iodide (61 mg, 0.165 mmol) and 1-bromopinacolone are mixed in 2-butanone (250 μL) and stirred under argon for 24 h at 60° C. After cooling to RT the mixture is filtered and the filtrate is purified by preparative HPLC through an RP phase. The combined fractions containing the desired product or Boc monocleavage product are combined, evaporated down, taken up in EA (15 mL), combined with conc. HCl (1 mL) and stirred for 4 h at RT. Then the mixture is evaporated to dryness.

Examples 36-41 are prepared analogously (Table 5).

TABLE 5

| # | structure | t_{Ret.} (HPLC) [min] | MS (ESI+) [M + H]⁺ |
|---|---|---|---|
| 35 | 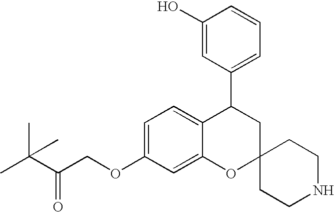 | 1.71 | 410 |
| 36 | 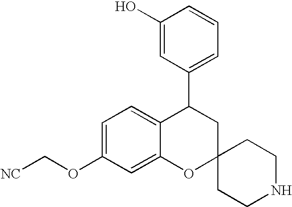 | 0.0 | 351 |
| 37 | 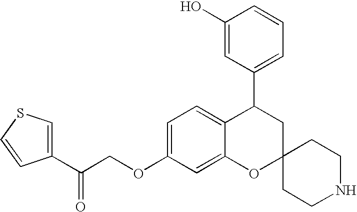 | 1.53 | 436 |
| 38 | 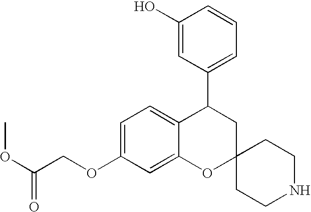 | 1.32 | 384 |
| 39 | 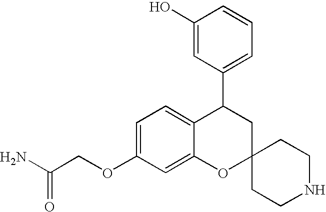 | 0.0 | 369 |
| 40 | 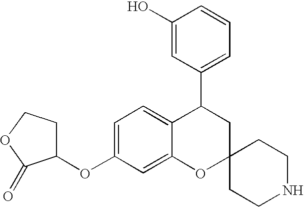 | 1.33 | 396 |
| 41 | 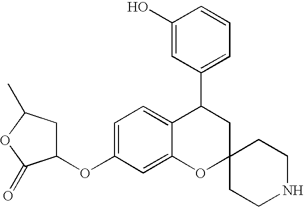 | 1.43 | 410 |

### Example 42

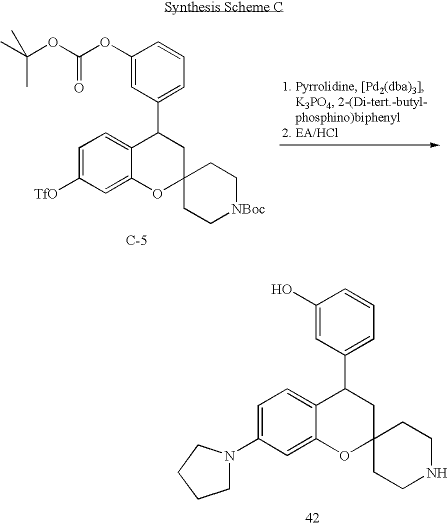

Under an argon atmosphere C-5 (152 mg, 0.236 mmol), tris-(dibenzylideneacetone)-dipalladium(0) (10.8 mg, 11.8 μmol), 2-(di-tert.-butyl-phosphino)biphenyl (10.7 mg, 35.4 μmol) and K₃PO₄ (72.4 mg, 330 μmol) are weighed out and suspended in DME (1 mL) in a 2 mL vial. The vial is closed and rinsed with argon through the septum. Then pyrrolidine (23.5 μL, 0.284 mmol) is added and it is stirred for 48 h at 80° C. After cooling to RT the mixture is divided between EA and saturated NH₄Cl soln., the organic phase is separated off and the aqueous phase is extracted again with EA. The combined organic extracts are dried on Na₂SO₄, filtered and evaporated down. The residue is purified by preparative HPLC. The combined fractions containing the desired product or Boc-monocleavage product are combined, evaporated down, taken up in EA (15 mL), combined with conc. HCl (1 mL) and stirred for 4 h at RT. Then the mixture is evaporated to dryness. Some of it is separated off by chiral HPLC (Chiracel O-DH).

Examples 43-55 are prepared analogously (Table 6).

### Example 56

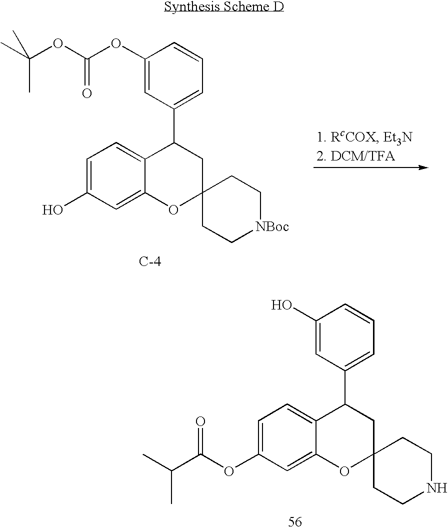

A solution of C-4 (70 mg, 137 μmol) in DCM (1 mL) and Et₃N (36.1 μL) is combined with isobutyric acid chloride (16.7 μL, 156 μmol) and stirred for 1 h at RT. The reaction mixture is divided between DCM and aqueous saturated NaHCO₃ soln. and the organic phase is dried and evaporated down. The residue is dissolved in DCM (10 mL), combined with trifluoroacetic acid (1 mL) and stirred for 2 h at RT. The reaction mixture is divided between DCM and aqueous saturated NaHCO₃ soln., the organic phase is dried and evaporated down. The residue is chromatographed through the RP-phase. The fractions containing the product are combined and evaporated down.

TABLE 6

| # | structure | t_{Ret.} (HPLC) [min] | MS (ESI+) [M + H]⁺ |
|---|---|---|---|
| 42 | 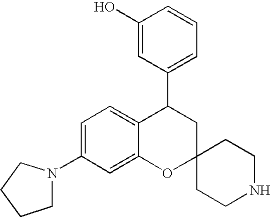 | 1.42 | 365 |
| 43 | 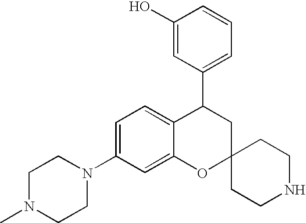 | 0.0 | 394 |
| 44 | 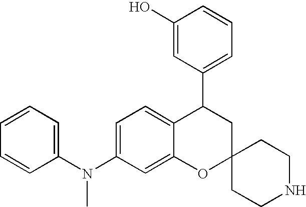 | 1.69 | 401 |
| 45 | 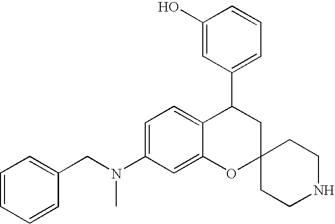 | 1.575 | 415 |
| 46 | 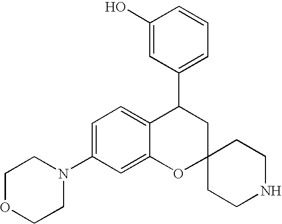 | 0.0 | 381 |
| 47 | 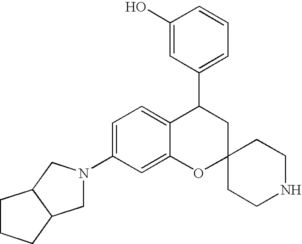 | 1.67 | 405 |
| 48 | 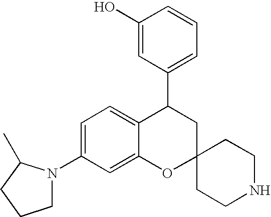 | 1.29 | 379 |
| 49 | 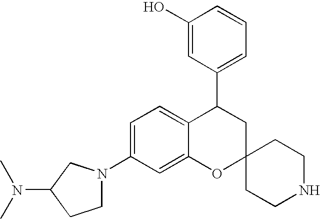 | 0.0 | 408 |
| 50 | 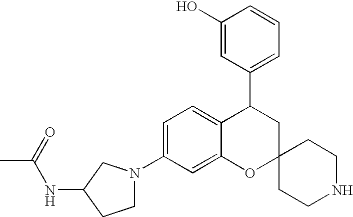 | 0.0 | 422 |
| 51 | 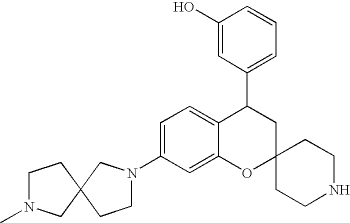 | 0.0 | 434 |
| 52 | 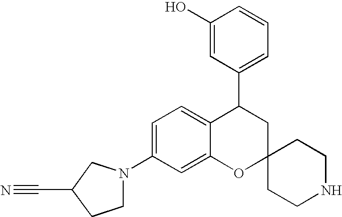 | 0.0 | 390 |
| 53 | 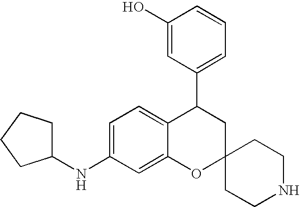 | 0.0 | 379 |
| 54 | 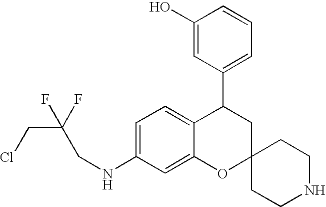 | 1.55 | 423 |
| 55* | 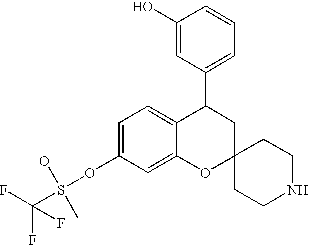 | 1.58 | 415 |
| 56 | 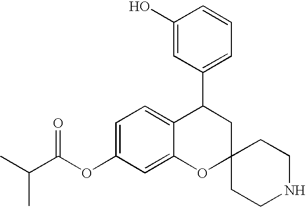 | 1.56 | 382 |

*Example 55 is formed directly from C-5 by cleaving the Boc protective groups in EA/conc. HCl without prior Buchwald-Hartwig reaction.

### Example 57

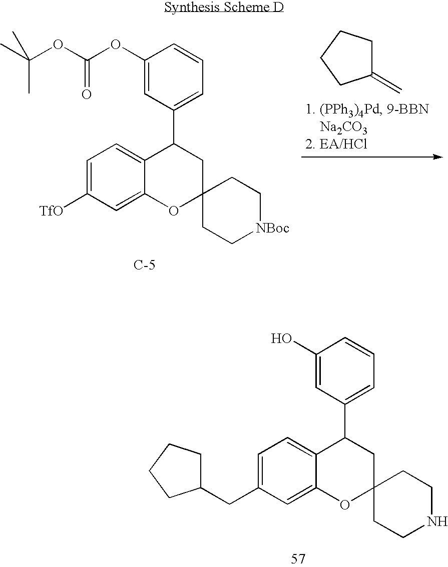

Methylenecyclopentane (20 μL, 0.186 mmol) is dissolved in dry THF (0.5 mL) under a protective gas atmosphere and at 0° C. combined with a solution of 9-BBN in THF (2.34 mL, 1.12 mmol). Then the mixture is stirred overnight at RT. The reaction mixture is carefully mixed with 1 mL H₂O, added dropwise to a mixture of C-5, tetrakis-(triphenyl-phosphine)-palladium(0) and Na₂CO₃ in 4 mL dioxane and refluxed overnight. After cooling to RT the mixture is divided between EA and NH₄Cl soln., the organic phase is separated off and the aqueous phase is again extracted with EA. The combined organic extracts are dried on Na₂SO₄, filtered and evaporated down. The residue is taken up in DMSO and chromatographed by RP preparative HPLC. The fraction containing the desired reaction product is evaporated down and the residue is dissolved in 5 mL EA, combined with 0.5 mL conc. HCl and stirred overnight at RT. After evaporating to dryness, the title compound is obtained.

### Example 58

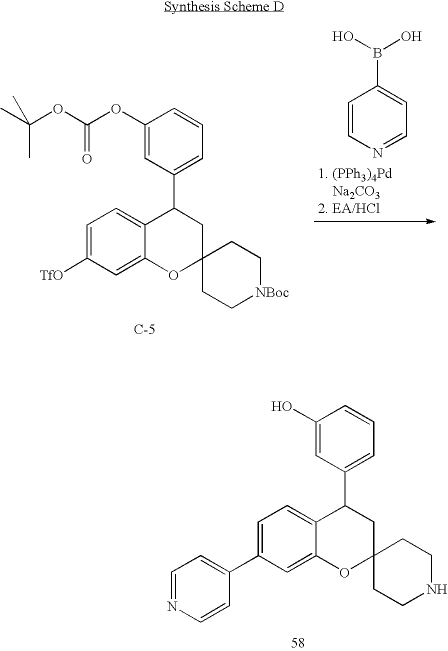

C-5 (50 mg, 77.7 μmol), pyridine-4-boric acid (14.3 mg, 116.5 μmol), tetrakis-(triphenylphosphine)-palladium(0) (9.0 mg, 7.8 μmol) and Na₂CO₃ (16.6 mg, 155.4 μmol) are suspended in dioxane/water (7 mL, 5:2) and stirred for 10 min at 150° C. under argon (microwave reactor). After cooling to RT the mixture is divided between EA and saturated NaCl soln., the organic phase is separated off and the aqueous phase is again extracted with EA. The combined organic extracts are dried on Na₂SO₄, filtered and evaporated down. The residue is taken up in DMSO and chromatographed by RP preparative HPLC. The fraction containing the desired reaction product is evaporated down and the residue is taken up in 5 mL EA, combined with 0.5 mL conc. HCl and stirred overnight at RT. After evaporation to dryness the title compound is obtained.

Examples 59 and 60 are prepared analogously (Table 7).

TABLE 7

| # | structure | t_{Ret.} (HPLC) [min]] | MS (ESI+) [M + H]⁺ |
|---|---|---|---|
| 57 | 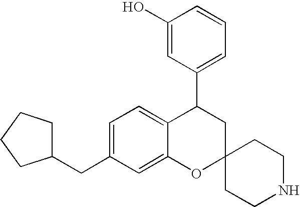 | 1.77 | 378 |
| 58 | 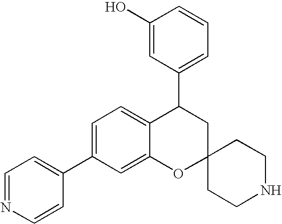 | 0.0 | 373 |
| 59 | 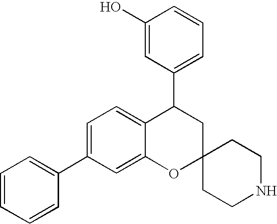 | 1.64 | 372 |
| 60 | 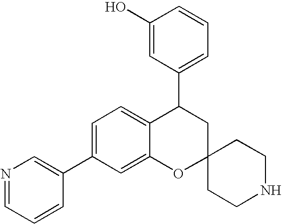 | 0.0 | 373 |

### Example 61

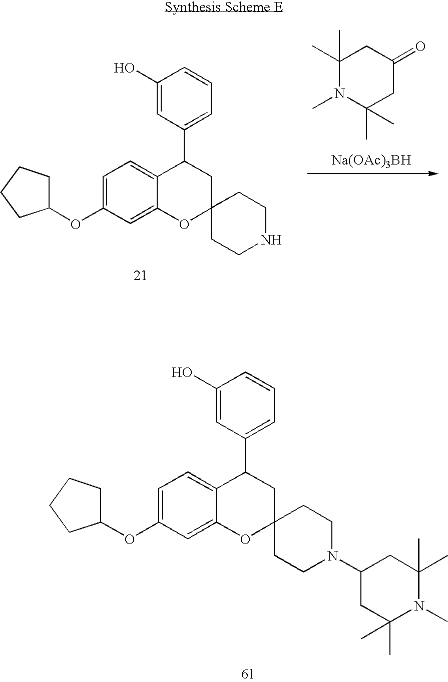

A suspension of 21 (20 mg, 0.053 mmol) and 1,2,2,6,6-pentamethyl-4-piperidone in DMA (150 μL) and acetic acid (5 μL) is stirred for 15 min under an argon atmosphere at RT and combined with Na(OAc)₃BH (34 mg, 0.16 mmol). After the reaction is complete, methanol is added, the solvents are eliminated in vacuo and the reaction mixture is purified by RP-chromatography (CH₃CN:H₂O, 5:95 to 95:5, 20 min, flow: 50 mL/min.).

Example 62 is synthesised analogously (Table 8).

### Example 63

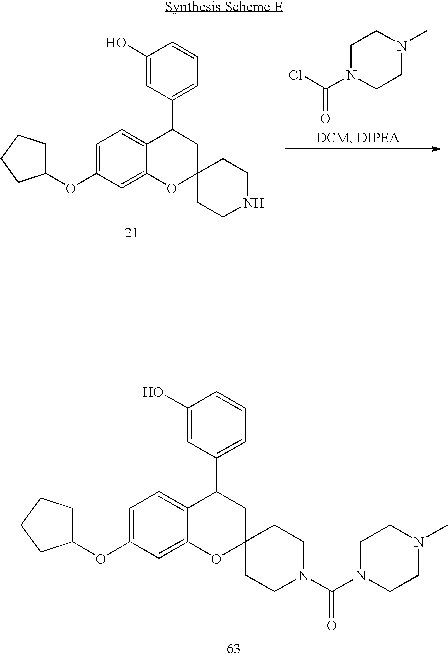

A suspension of 21 (20 mg, 0.053 mmol) and DIPEA (25.6 μL, 0.16 mmol) in DCM is combined at 0° C. with 4-methylpiperazine-1-carbonyl chloride hydrochloride (10.9 mg, 0.05 mmol) and stirred at RT. After the reaction is complete the solvents are eliminated in vacuo and the residue is purified by preparative RP chromatography. (CH₃CN:H₂O, 5:95 to 95:5, 15 min, 50 mL/min).

Example 64 is synthesised analogously (Table 8).

TABLE 8

| # | structure | t_{Ret.} (HPLC) [min]] | MS (ESI+) [M + H]⁺ |
|---|---|---|---|
| 61 | 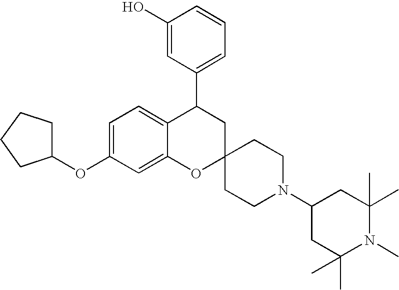 | 1.46 | 533 |
| 62 | 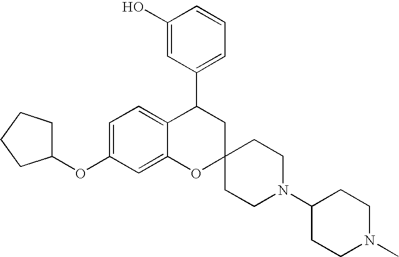 | 1.39 | 477 |
| 63 | 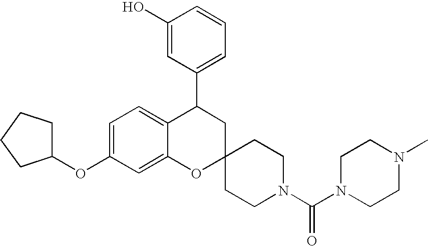 | 1.70 | 506 |
| 64 | 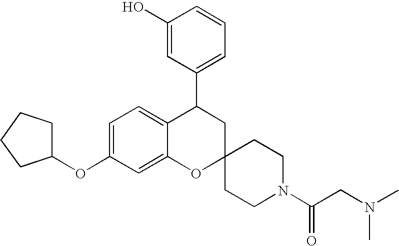 | 1.71 | 465 |

The following Examples describe the biological activity of the compounds according to the invention without restricting the invention to these Examples.

As has been found, the compounds of general formula (1) are characterised by their wide range of applications in the therapeutic field. Particular mention should be made of those applications in which the inhibition of specific cell cycle kinases, particularly the inhibiting effect on the proliferation of cultivated human tumour cells but also the proliferation of other cells, such as endothelial cells, for example, plays a part.

As demonstrated by DNA staining followed by Cellomics Array Scan analysis (Cellcycle Analysis), the inhibition of proliferation brought about by the compounds according to the invention is mediated above all by the defective formation of bipolar mitotic spindles. As a result the duplicated chromosomes cannot be correctly divided into two daughter cells, leading finally to inhibition of proliferation and apoptosis.

Measurement of the inhibition of proliferation on cultivated human tumour cells To measure proliferation on cultivated human tumour cells, cells of colon carcinoma cell line HCT 116 (American Type Culture Collection (ATCC)) are cultivated in RPMI 1640 medium (Gibco) and 10% fetal calf serum (Gibco). Then the HCT 116 cells are placed in 96-well flat-bottomed plates (Falcon) at a density of 1400 cells per well in RPMI 1640 medium and incubated overnight in an incubator (at 37° C. and 5% CO₂). The active substances are added to the cells in various concentrations. After 72 hours incubation 20 μl AlamarBlue reagent (AccuMed International) is added to each well, and the cells are incubated for a further 3-4 hours. After incubation the colour change of the AlamarBlue reagent is determined in a Wallac Microbeta fluorescence spectrophotometer. EC₅₀ values are calculated using Standard Levenburg Marquard algorithms (GraphPadPrizm). Most of the compounds of Examples 1 to 64 exhibit good to very good activity in the above inhibition test, i.e. an EC₅₀ value of less than 5 μmol, generally less than 1 μmol. Correspondingly, the compounds according to the invention are also tested on other tumour cells. For example these compounds are actively tested on carcinomas of all kinds of tissue [e.g. lung (NCI-H460) and prostate (PC-3)] and may be used for such indications. This demonstrates the broad range of uses of the compounds according to the invention for treating all kinds of tumours.

### Cellomics Array Scan

NCI-H460 cells are seeded into fibronectin-coated 96-well dishes (BD BioCoat) in RPMI 1640 medium (Gibco) with 10% fetal calf serum (Gibco) in a density of 4000 cells per well and incubated overnight in an incubator (at 37° C. and 5% CO₂). The active substances are added to the cells in various concentrations. After 24 h incubation the cells are fixed for 10 min by the addition of 100 μL with 7.4% formaldehyde solution at RT, and washed twice with PBS solution. Then the cells are permeabilised by the addition of 100 μL of 0.1% Triton X100 in PBS for 90 seconds, the permeabilising solution is removed by suction filtering and washed with PBS. Non-specific binding sites are saturated by incubating for 20 min with blocking solution (10% Normal Goat Serum in 2% BSA/PBS). After a washing step with PBS, antibodies against phosphorylated histone H3 (1:500 diluted, Up-state) or against tubulin (1:1000 diluted, Sigma) in 2% BSA/PBS are added and the mixture is incubated for 60 min, washed twice with 0.01% Tween/PBS and incubated for 1 h with Alexa 488-Goat anti Mouse (diluted 1:1000), Alexa 594-Goat anti Rabbit (diluted 1:5000) and 4′,6-diamidino-2-phenylindole (DAPI, final concentration 300 nM, Molecular Probes) in 2% BSA/PBS in the dark. After washing twice with 0.01% Tween/PBS and a washing step with PBS the wells are filled with 270 μL of PBS, stuck down with black adhesive film and analysed in the Array Scan of Cellomics. For this, the DNA content of the cells is determined and the cell cycle arrest phase is established. In parallel, analysis of the spindle shape and the content of phosphorylated histone H3 allows a more precise assessment of the cell cycle arrest to be made.

On the basis of their biological properties the new compounds of general formula (1), the isomers thereof, pharmacologically acceptable salts and polymorphs thereof are suitable for treating diseases characterised by excessive or abnormal cell proliferation.

Such diseases include for example: viral infections (e.g. HIV and Kaposi's sarcoma); inflammatory and autoimmune diseases (e.g. colitis, arthritis, Alzheimer's disease, glomerulonephritis and wound healing); bacterial, fungal and/or parasitic infections; leukaemias, lymphomas and solid tumours (e.g. carcinomas and sarcomas), skin diseases (e.g. psoriasis); diseases based on hyperplasia which are characterised by an increase in the number of cells (e.g. fibroblasts, hepatocytes, bones and bone marrow cells, cartilage or smooth muscle cells or epithelial cells (e.g. endometrial hyperplasia)); bone diseases and cardiovascular diseases (e.g. restenosis and hypertrophy). They are also useful for protecting proliferating cells (e.g. hair, intestinal, blood and progenitor cells) from DNA damage caused by radiation, UV treatment and/or cytostatic treatment.

For example, the following cancers may be treated with compounds according to the invention, without being restricted thereto: brain tumours such as for example acoustic neurinoma, astrocytomas such as pilocytic astrocytomas, fibrillary astrocytoma, protoplasmic astrocytoma, gemistocytary astrocytoma, anaplastic astrocytoma and glioblastoma, brain lymphomas, brain metastases, hypophyseal tumour such as prolactinoma, HGH (human growth hormone) producing tumour and ACTH producing tumour (adrenocorticotropic hormone), craniopharyngiomas, medulloblastomas, meningeomas and oligodendrogliomas; nerve tumours (neoplasms) such as for example tumours of the vegetative nervous system such as neuroblastoma sympathicum, ganglioneuroma, paraganglioma (pheochromocytoma, chromaffinoma) and glomus-caroticum tumour, tumours on the peripheral nervous system such as amputation neuroma, neurofibroma, neurinoma (neurilemmoma, Schwannoma) and malignant Schwannoma, as well as tumours of the central nervous system such as brain and bone marrow tumours; intestinal cancer such as for example carcinoma of the rectum, colon, anus, small intestine and duodenum; eyelid tumours such as basalioma or basal cell carcinoma; pancreatic cancer or carcinoma of the pancreas; bladder cancer or carcinoma of the bladder; lung cancer (bronchial carcinoma) such as for example small-cell bronchial carcinomas (oat cell carcinomas) and non-small cell bronchial carcinomas such as plate epithelial carcinomas, adenocarcinomas and large-cell bronchial carcinomas; breast cancer such as for example mammary carcinoma such as infiltrating ductal carcinoma, colloid carcinoma, lobular invasive carcinoma, tubular carcinoma, adenocystic carcinoma and papillary carcinoma; non-Hodgkin's lymphomas (NHL) such as for example Burkitt's lymphoma, low-malignancy non-Hodgkin's lymphomas (NHL) and mucosis fungoides; uterine cancer or endometrial carcinoma or corpus carcinoma; CUP syndrome (Cancer of Unknown Primary); ovarian cancer or ovarian carcinoma such as mucinous, endometrial or serous cancer; gall bladder cancer; bile duct cancer such as for example Klatskin tumour; testicular cancer such as for example seminomas and non-seminomas; lymphoma (lymphosarcoma) such as for example malignant lymphoma, Hodgkin's disease, non-Hodgkin's lymphomas (NHL) such as chronic lymphatic leukaemia, leukaemic reticuloendotheliosis, immunocytoma, plasmocytoma (multiple myeloma), immunoblastoma, Burkitt's lymphoma, T-zone mycosis fungoides, large-cell anaplastic lymphoblastoma and lymphoblastoma; laryngeal cancer such as for example tumours of the vocal cords, supra-glottal, glottal and subglottal laryngeal tumours; bone cancer such as for example osteochondroma, chondroma, chondroblastoma, chondromyxoid fibroma, osteoma, osteoid osteoma, osteoblastoma, eosinophilic granuloma, giant cell tumour, chondrosarcoma, osteosarcoma, Ewing's sarcoma, reticulo-sarcoma, plasmocytoma, giant cell tumour, fibrous dysplasia, juvenile bone cysts and aneurysmatic bone cysts; head and neck tumours such as for example tumours of the lips, tongue, floor of the mouth, oral cavity, gums, palate, salivary glands, throat, nasal cavity, paranasal sinuses, larynx and middle ear; liver cancer such as for example liver cell carcinoma or hepatocellular carcinoma (HCC); leukaemias, such as for example acute leukaemias such as acute lymphatic/lymphoblastic leukaemia (ALL), acute myeloid leukaemia (AML); chronic leukaemias such as chronic lymphatic leukaemia (CLL), chronic myeloid leukaemia (CML); stomach cancer or gastric carcinoma such as for example papillary, tubular and mucinous adenocarcinoma, signet ring cell carcinoma, adenosquamous carcinoma, small-cell carcinoma and undifferentiated carcinoma; melanomas such as for example superficially spreading, nodular, lentigo-maligna and acral-lentiginous melanoma; renal cancer such as for example kidney cell carcinoma or hypernephroma or Grawitz's tumour; oesophageal cancer or carcinoma of the esophagus; penile cancer; prostate cancer; throat cancer or carcinomas of the pharynx such as for example nasopharynx carcinomas, oropharynx carcinomas and hypopharynx carcinomas; retinoblastoma; vaginal cancer or vaginal carcinoma; plate epithelial carcinomas, adenocarcinomas, in situ carcinomas, malignant melanomas and sarcomas; thyroid carcinomas such as for example papillary, follicular and medullary thyroid carcinoma, as well as anaplastic carcinomas; spinalioma, epidormoid carcinoma and plate epithelial carcinoma of the skin; thymomas, cancer of the urethra and cancer of the vulva. The new compounds may be used for the prevention, short-term or long-term treatment of the above-mentioned diseases, optionally also in combination with radiotherapy or other “state-of-the-art” compounds, such as e.g. cytostatic or cytotoxic substances, cell proliferation inhibitors, anti-angiogenic substances, steroids or antibodies.

The compounds of general formula (1) may be used on their own or in combination with other active substances according to the invention, optionally also in combination with other pharmacologically active substances.

Chemotherapeutic agents which may be administered in combination with the compounds according to the invention include, without being restricted thereto, hormones, hormone analogues and antihormones (e.g. tamoxifen, toremifene, raloxifene, fulvestrant, megestrol acetate, flutamide, nilutamide, bicalutamide, aminoglutethimide, cyproterone acetate, finasteride, buserelin acetate, fludrocortisone, fluoxymesterone, medroxyprogesterone, octreotide), aromatase inhibitors (e.g. anastrozole, letrazole, liarozole, vorazole, exemestane, atamestane), LHRH agonists and antagonists (e.g. goserelin acetate, luprolide), inhibitors of growth factors (growth factors such as for example “platelet derived growth factor” and “hepatocyte growth factor”, inhibitors are for example “growth factor” antibodies, “growth factor receptor” antibodies and tyrosinekinase inhibitors, such as for example gefitinib, imatinib, lapatinib and trastuzumab); antimetabolites (e.g. antifolates such as methotrexate, raltitrexed, pyrimidine analogues such as 5-fluorouracil, capecitabin and gemcitabin, purine and adenosine analogues such as mercaptopurine, thioguanine, cladribine and pentostatin, cytarabine, fludarabine); antitumour antibiotics (e.g. anthracyclins such as doxorubicin, daunorubicin, epirubicin and idarubicin, mitomycin-C, bleomycin, dactinomycin, plicamycin, streptozocin); platinum derivatives (e.g. cisplatin, oxaliplatin, carboplatin); alkylation agents (e.g. estramustin, meclorethamine, melphalan, chlorambucil, busulphan, dacarbazin, cyclophosphamide, ifosfamide, temozolomide, nitrosoureas such as for example carmustin and lomustin, thiotepa); antimitotic agents (e.g. Vinca alkaloids such as for example vinblastine, vindesin, vinorelbin and vincristine; and taxanes such as paclitaxel, docetaxel); topoisomerase inhibitors (e.g. epipodophyllotoxins such as for example etoposide and etopophos, teniposide, amsacrin, topotecan, irinotecan, mitoxantron) and various chemotherapeutic agents such as amifostin, anagrelid, clodronat, filgrastin, interferon alpha, leucovorin, rituximab, procarbazine, levamisole, mesna, mitotane, pamidronate and porfimer.

Suitable preparations include for example tablets, capsules, suppositories, solutions,—particularly solutions for injection (s.c., i.v., i.m.) and infusion—elixirs, emulsions or dispersible powders. The content of the pharmaceutically active compound(s) should be in the range from 0.1 to 90 wt.-%, preferably 0.5 to 50 wt.-% of the composition as a whole, i.e. in amounts which are sufficient to achieve the dosage range specified below. The doses specified may, if necessary, be given several times a day.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection and infusion are prepared in the usual way, e.g. with the addition of isotonic agents, preservatives such as p-hydroxybenzoates, or stabilisers such as alkali metal salts of ethylenediamine tetraacetic acid, optionally using emulsifiers and/or dispersants, whilst if water is used as the diluent, for example, organic solvents may optionally be used as solvating agents or dissolving aids, and transferred into injection vials or ampoules or infusion bottles.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

Excipients which may be used include, for example, water, pharmaceutically acceptable organic solvents such as paraffins (e.g. petroleum fractions), vegetable oils (e.g. groundnut or sesame oil), mono- or polyfunctional alcohols (e.g. ethanol or glycerol), carriers such as e.g. natural mineral powders (e.g. kaolins, clays, talc, chalk), synthetic mineral powders (e.g. highly dispersed silicic acid and silicates), sugars (e.g. cane sugar, lactose and glucose) emulsifiers (e.g. lignin, spent sulphite liquors, methylcellulose, starch and polyvinylpyrrolidone) and lubricants (e.g. magnesium stearate, talc, stearic acid and sodium lauryl sulphate).

The preparations are administered by the usual methods, preferably by oral or transdermal route, most preferably by oral route. For oral administration the tablets may, of course contain, apart from the abovementioned carriers, additives such as sodium citrate, calcium carbonate and dicalcium phosphate together with various additives such as starch, preferably potato starch, gelatine and the like. Moreover, lubricants such as magnesium stearate, sodium lauryl sulphate and talc may be used at the same time for the tabletting process. In the case of aqueous suspensions the active substances may be combined with various flavour enhancers or colourings in addition to the excipients mentioned above.

For parenteral use, solutions of the active substances with suitable liquid carriers may be used.

The dosage for intravenous use is from 1-1000 mg per hour, preferably between 5 and 500 mg per hour.

However, it may sometimes be necessary to depart from the amounts specified, depending on the body weight, the route of administration, the individual response to the drug, the nature of its formulation and the time or interval over which the drug is administered. Thus, in some cases it may be sufficient to use less than the minimum dose given above, whereas in other cases the upper limit may have to be exceeded. When administering large amounts it may be advisable to divide them up into a number of smaller doses spread over the day.

The formulation examples that follow illustrate the present invention without restricting its scope:

### Examples of Pharmaceutical Formulations

### A)

| Tablets | per tablet |
|---|---|
| active substance according to formula (1) | 100 mg |
| lactose | 140 mg |
| corn starch | 240 mg |
| polyvinylpyrrolidone | 15 mg |
| magnesium stearate | 5 mg |
| | 500 mg |

The finely ground active substance, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

### B)

| Tablets | per tablet |
|---|---|
| active substance according to formula (1) | 80 mg |
| lactose | 55 mg |
| corn starch | 190 mg |
| microcrystalline cellulose | 35 mg |
| polyvinylpyrrolidone | 15 mg |
| sodium-carboxymethyl starch | 23 mg |
| magnesium stearate | 2 mg |
| | 400 mg |

The finely ground active substance, some of the corn starch, lactose, microcrystalline cellulose and polyvinylpyrrolidone are mixed together, the mixture is screened and worked with the remaining corn starch and water to form a granulate which is dried and screened. The sodiumcarboxymethyl starch and the magnesium stearate are added and mixed in and the mixture is compressed to form tablets of a suitable size.

### C)

Ampoule solution

| | | |
|---|---|---|
| active substance according to formula (1) | 50 | mg |
| sodium chloride | 50 | mg |
| water for inj. | 5 | ml |

The active substance is dissolved in water at its own pH or optionally at pH 5.5 to 6.5 and sodium chloride is added to make it isotonic. The solution obtained is filtered free from pyrogens and the filtrate is transferred under aseptic conditions into ampoules which are then sterilised and sealed by fusion. The ampoules contain 5 mg, 25 mg and 50 mg of active substance.

1. Compounds of general formula (1) 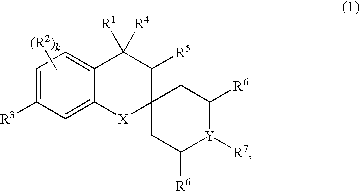
wherein
X denotes —O—, —S—, —SO— or —SO₂— and
Y denotes N or CH,
R¹ is selected from among C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, all the above-mentioned groups optionally being substituted by one or more identical or different R^{a} and/or R^{b},
R² and R³ are each independently of one another selected from among R^{a} and R^{b}, or
R³ together with an adjacent R² in the ortho position and the two carbon atoms to which R² and R³ are fixed, may form a phenyl ring, a 5-6 membered heteroaryl, 5-7 membered cycloalkyl or 5-7 membered heterocycloalkyl, while the above-mentioned ring systems may optionally be substituted by one or more identical or different R^{a} and/or R^{b},
R⁴ denotes hydrogen, C₁₋₆alkyl or C₁₋₆haloalkyl, optionally substituted by one or more identical or different groups —OR^{h} and/or —NR^{h}R^{h},
R⁵ is selected from among hydrogen, C₁₋₆haloalkyl, halogen, —CN, —C(O)OR^{h}, —C(O)NR K and C₁₋₆alkyl, the latter optionally being substituted by one or more identical or different groups —OR^{h},
each R⁶ is selected independently of one another from among hydrogen, C₁₋₆alkyl, C₆₋₁₀aryl and halogen,
R⁷, in the event that Y denotes N, is selected from among hydrogen, R^{a}, —OR³, —NR^{a}R^{a}, —S(O)R^{a}, —S(O)NR^{a}R^{a}, —S(O)₂R^{a}, —S(O)₂OR^{a}, —S(O)₂NR^{a}R^{a}, —[S(O)₂]₂R^{a}, —S(O)OR^{a}, —C(O)R^{a}, —C(S)R^{a}, —N(R^{g})C(O)R^{a}, —C(NOH)R^{a}, —C(NR^{g})R^{a}, —C(O)OR^{a}, —C(O)SR³, —C(O)NR^{a}R^{a}, —C(S)NR^{a}R^{a}, C(O)N(R^{g})NR^{a}R^{a}, —N(R^{g})C(O)NR^{a}R^{a}, —C(NR^{g})OR^{a}, —C(NR^{g})SR^{a}, —C(NR^{g})NR^{a}R^{a}, —C(O)N(R^{g})C(O)R^{a}, —[C(O)]₂R^{a}, —[C(O)]₂OR^{a}, —[C(O)]₂NR^{a}R^{a} and —C(O)N(R^{g})C(O)OR³, or
R⁷, in the event that Y denotes CH, is selected from among 2-6 membered heteroalkyl, 5-12 membered heteroaryl, 3-14 membered heterocycloalkyl, all the above-mentioned groups optionally being substituted by one or more identical or different R^{a} and/or R^{b}, as well as —NR^{a}R^{a}, —N(OR^{a})R^{a}, —N(R^{g})NR^{a}R^{a}, —N(R^{g})S(O)R^{a}, —N(R^{g})S(O)₂R^{a}, —N[S(O)₂R^{a}]₂, —N(R^{g})S(O)₂OR^{a}, —N(R^{g})S(O)₂NR^{a}R^{a}, —N(R^{g})S(O)OR^{a}, —N(R^{g})S(O)NR^{a}R^{a}, —N(R^{g})C(O)R^{a}, —N[C(O)R]₂, —N(R^{g})C(S)R^{a}, —N[C(O)R^{a}]NR^{a}R^{a}, N(R^{g})N(R^{g})C(O)R^{a}, —N(OR^{g})C(O)R^{a}, —N(R^{g})C(NOH)R^{a}, —N(R^{g})C(NR^{g})R^{a}, —N(R^{g})C(O)OR^{a}, —N(R^{g})C(O)SR³, —N(R^{g})C(O)NR^{a}R^{a}, N(R^{g})C(S)NR^{a}R^{a}, —N(R^{g})C(O)NR^{g}NR^{a}R^{a}, —N(R^{g})N(R^{g})C(O)NR^{a}R^{a}, —N(R^{g})C(NR^{g})OR^{a}, —N(R^{g})C(NR^{g})SR^{a}, —N(R^{g})C(NR^{g})NR^{a}R^{a}, —[N(R^{g})C(O)]₂R^{a}, —N(R^{g})[C(O)]₂R^{a}, —N {[C(O)]₂R^{a}}₂, —N(R^{g})[C(O)]₂OR^{a}, —N(R^{g})[C(O)]₂NR^{a}R^{a}, —N {[C(O)]₂OR^{a}}₂, —N {[C(O)]₂NR^{a}R^{a}}₂ and —[N(R^{g})C(O)]₂OR^{a},
k denotes either 0, 1, 2 or 3,
each R^{a} independently of one another denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
each R^{b} denotes a suitable group and is selected independently of one another from among ═O, —OR^{c}, C₁₋₃haloalkyloxy, —OCF₃, ═S, —SR^{c}, ═NR^{c}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g}) [S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})C(O)]₂R^{c}, —N(R^{g})[C(O)]₂R^{c}, —N {[C(O)]₂R^{c}}₂, —N(R^{g})[C(O)]₂OR^{c}, —N(R^{g})[C(O)]₂NR^{c}R^{c}, —N {[C(O)]₂OR^{c}}₂, —N {[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c},
each R^{c} independently of one another denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{d} denotes a suitable group and is selected independently of one another from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}. —C(O)N(R^{g})OR^{e}, —C(NR^{g})NR^{e}R^{e}, —C(NOH)R^{e}, —C(NOH)NR^{e}R^{e}, —OC(O)R^{e}—OC(O)OR^{e} OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R^{g})C(NR^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e} N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g}) [C(O)]₂R^{e}, —N {[C(O)]₂R^{e}}₂, —N(R^{g})[C(O)]₂—R^{e}, —N(R^{g})[C(O)]₂NR^{e}R^{e}, —N{[C(O)]₂OR^{e}}₂, —N {[C(O)]₂NR^{e}R^{e}}₂, —[N(R^{g})C(O)]₂OR^{e}, —N(R^{g})C(NR^{g})OR^{e}, —N(R^{g})C(NOH)R^{e}, —N(R^{g})C(NR^{g})SR^{e} and —N(R^{g})C(NR^{g})NR^{e}R^{e},
each R^{e} independently of one another denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{f} denotes a suitable group and in each case is selected independently of one another from among ═O, —OR^{g}, C₁₋₃haloalkyloxy, —OCF₃, ═S, —SR^{g}, ═NR^{g}, ═NOR^{g}, ═NNR^{g}R^{g}, ═NN(R^{h})C(O)NR^{g}R^{g}, —NR^{g}R^{g}, —ONR^{g}R^{g}, —N(R^{e})NR^{g}R^{g}, halogen, —CF₃, —CN, —NC, —OCN, —SCN, —NO, —NO₂, ═N₂, —N₃, —S(O)R^{g}, —S(O)OR^{g}, —S(O)₂R^{g}, —S(O)₂OR^{g}, —S(O)NR^{g}R^{g}, —S(O)₂NR^{g}R^{g}, —OS(O)R^{g}, —OS(O)₂R^{g}, —OS(O)₂OR^{g}, —OS(O)NR^{g}R^{g}, —OS(O)₂NR^{g}R^{g}, —C(O)R^{g}, —C(O)OR^{g}, —C(O)SR^{g}, —C(O)NR^{g}R^{g}, —C(O)N(R^{h})NR^{g}R^{g}, —C(O)N(R^{e})OR^{g}, —C(NR^{h})NR^{g}R^{g}, —C(NOH)R^{g}, —C(NOH)NR^{g}R^{g}, —OC(O)R^{g}, —OC(O)OR^{g}, —OC(O)SR^{g}, —OC(O)NR^{g}R^{g}, —OC(NR^{h})NR^{g}R^{g}, —SC(O)R^{g}, —SC(O)OR^{g}, —SC(O)NR^{g}R^{g}, —SC(R^{h})NR^{g}R^{g}, —N(R^{h})C(O)R^{g}, —N[C(O)R^{g}]₂, —N(OR^{h})C(O)R^{g}, —N(R^{h})C(NR^{h})R^{g}, —N(R^{h})N(R^{h})C(O)R^{g}, —N[C(O)R^{g}]NR^{g}R^{g}, —N(R^{h})C(S)R^{g}, —N(R^{h})S(O)R^{g}, —N(R^{h})S(O)OR^{g}, —N(R^{h})S(O)₂R^{g}, —N[S(O)₂R^{g}]₂, —N(R^{h})S(O)₂OR^{g}, —N(R^{e})S(O)₂NR^{g}R^{g}, —N(e) [S(O)₂]₂R^{g}, —N(R^{h})C(O)OR^{g}, —N(R^{h})C(O)SR^{g}, —N(R^{h})C(O)NR^{g}R^{g}, —N(R^{h})C(O)NR^{h}NR^{g}R^{g}, —N(R^{h})N(R^{h})C(O)NR^{g}R^{g}, —N(R^{h})C(S)NR^{g}R^{g}, —[N(R^{h}) C(O)]₂R^{g}, —N {[C(O)]₂R^{g} }₂, —N(R^{h})[C(O)]₂OR^{g}, —N(R^{h})[C(O)]₂NR^{g}R^{g}, —N {[C(O)]₂OR^{g}}₂, —N {[C(O)]₂NR^{g}R^{g} }₂, —[N(R^{h})C(O)]₂OR^{g}, —N(R^{h})C(NR^{h})OR^{g}, —N(R^{h})C(NOH)R^{g}, —N(R^{h})C(NR^{h})SR^{g} and —N(R^{h})C(NR^{h})NR^{g}R^{g},
each R^{g} independently of one another denotes hydrogen or a group optionally substituted by one or more identical or different R^{h}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{h} is selected independently of one another from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
optionally in the form of the tautomers, the racemates, the enantiomers, the diastereomers and the mixtures thereof, and optionally the pharmacologically acceptable salts thereof,
with the proviso that the compounds
4-(7,8-dihydro-6H-[1,3]dioxolo[4,5-g]spiro[chromene-2,1′-cyclohexan]-8-yl)-methoxyphenyl;
methyl-1-(3,4-dihydrospiro[chromene-2,1′-cyclohexan]-4-yl)-1H-imidazole-5-carboxylate;
ethyl-1-(5-chloro-7-methoxy-3,4-dihydrospiro[chromene-2,1′-cyclohexan]-4-yl)-1H-imidazole-5-carboxylate;
ethyl-1-(5-chloro-7-methoxy-3,4-dihydrospiro[chromene-2,1′-cyclohexan]-4-yl)-2-mercapto-1H-imidazole-5-carboxylate;
methyl-1-(3,4-dihydrospiro[chromene-2,1′-cyclohexan]-4-yl)-2-mercapto-1H-imidazole-5-carboxylate;
1-(3,4-dihydrospiro[chromene-2,1′-cyclohexan]-4-yl)-1H-imidazole-5-carboxylic acid;
ethyl-1-(3′,4′-dihydrospiro[cyclohexane-1,2′-thiochromen]-4′-yl)-1H-imidazole-5-carboxylate;
ethyl-1-(3′,4′-dihydrospiro[cyclohexane-1,2′-thiochromen]-4′-yl)-2-mercapto-1H-imidazole-5-carboxylate;
ethyl-1-(1′-oxido-3′,4′-dihydrospiro[cyclohexane-1,2′-thiochromen]-4′-yl)-1H-imidazole-5-carboxylate;
ethyl-2-mercapto-1-(1′-oxido-3′,4′-dihydrospiro[cyclohexane-1,2′-thiochromen]-4′-yl)-1H-imidazole-5-carboxylate;
ethyl-1-(1′,1′-dioxido-3′,4′-dihydrospiro[cyclohexane-1,2′-thiochromen]-4′-yl)-1H-imidazole-5-carboxylate;
ethyl-1-(1′,1′-dioxido-3′,4′-dihydrospiro[cyclohexane-1,2′-thiochromen]-4′-yl)-2-mercapto-1H-imidazole-5-carboxylate;
1-(3,4-dihydrospiro[chromene-2,1′-cyclohexan]-4-yl)-5-(methoxycarbonyl)-3-methyl-1H-imidazol-3-ium;
methyl-1-oxy-3-(3,4-dihydrospiro[chromene-2,1′-cyclohexan]-4-yl)-3H-imidazol-4-carboxylate;
4-(3,4-dihydrospiro[chromene-2,4′-piperidin]-4-ylmethyl)-N,N-diethylbenzamide;
3-hydroxy-4-(6-fluoro-3,4-dihydrospiro[chromene-2,4′-piperidin]-4-yl)-N,N-diethyl-benzamide;
3-hydroxy-4-(3,4-dihydrospiro[chromene-2,4′-piperidin]-4-yl)-N,N-diethylbenzamide;
5-(5-methoxy-3,4-dihydrospiro[chromene-2,4′-piperidin]-4-yl)-N,N-diethylpyridine-2-carboxamide;
4-(5-methoxy-3,4-dihydrospiro[chromene-2,4′-piperidin]-4-yl)-N,N-diethylbenzamide;
4-(3,4-dihydrospiro[chromene-2,4′-piperidin]-4-yl)-N,N-diethylbenzamide;
4-(6-fluoro-3,4-dihydrospiro[chromene-2,4′-piperidin]-4-yl)-N,N-diethylbenzamide;
4-(6-cyclopropylmethoxy-3,4-dihydrospiro[chromene-2,4′-piperidin]-4-yl)-N,N-diethylbenzamide;
4-(3,4-dihydrospiro[chromene-2,4′-piperidin]-4-yl)-N,N-diethylpyridine-2-carboxamide;
4-(6-fluoro-3,4-dihydrospiro[chromene-2,4′-piperidin]-4-yl)-N,N-diethylpyridine-2-carboxamide;
4-(6,7-dimethyl-3,4-dihydrospiro[chromene-2,4′-piperidin]-4-yl)-N,N-diethylpyridine-2-carboxamide;
4-(6-hydroxy-3,4-dihydrospiro[chromene-2,4′-piperidin]-4-yl)-N,N-diethylbenzamide;
4-(5-hydroxy-3,4-dihydrospiro[chromene-2,4′-piperidin]-4-yl)-N,N-diethylbenzamide;
4-(6-methyl-3,4-dihydrospiro[chromene-2,4′-piperidin]-4-yl)-N,N-diethylbenzamide;
{3,4-dihydro-4-(4-methylphenyl)spiro[chromene-2,4′-piperidin]-1′-yl}-acetic acid;
ethyl-{3,4-dihydro-4-(4-methylphenyl)spiro[chromene-2,4′-piperidin]-1′-yl}-acetate;
{3,4-dihydro-4-(4-fluorophenyl)spiro[chromene-2,4′-piperidin]-1′-yl}-acetic acid;
ethyl-{3,4-dihydro-4-(4-fluorophenyl)spiro[chromene-2,4′-piperidin]-1′-yl}-acetate and the compounds with the structures (i) and (ii) 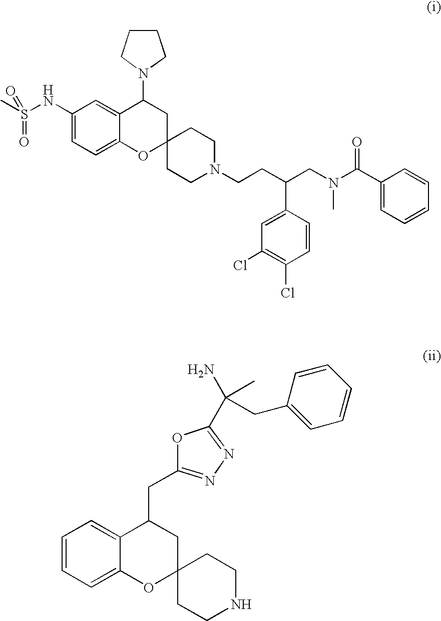
are excluded.

2. Compounds according to claim 1, wherein
X denotes oxygen and
R⁵ and R⁶ denote hydrogen.

3. Compounds according to claim 1, wherein
R¹ denotes C₆₋₁₀aryl or 5-12 membered heteroaryl, optionally substituted by one or more identical or different R^{a} and/or R^{b}, and
R^{a} and R^{b} are as hereinbefore defined.

4. Compounds according to claim 3, wherein
in the event that R¹ is substituted by one or two R^{b} and not by R^{a}, none of these R^{b} may be —C(O)NR^{c}R^{c} and
R¹, R^{a} and R^{c} are as hereinbefore defined.

5. Compounds according to claim 1,
wherein
Y denotes nitrogen.

6. Compounds according to claim 1,
wherein
R¹ denotes C₆₋₁₀aryl or 5-12 membered heteroaryl, optionally substituted by one or more identical or different groups, selected from among —OR^{c} and halogen, and
R⁴ denotes hydrogen and
R^{c} is as hereinbefore defined.

7. Compounds according to claim 1,
wherein
R³ is not hydrogen.

8. Compounds according to claim 1,
wherein
R³ is selected from among —OR^{c}, —NR^{c}R^{c} and 3-14 membered heterocycloalkyl, the latter optionally being substituted by one or more identical or different R^{b} and/or R^{c} and
R^{b} and R^{c} are as hereinbefore defined.

9. Compounds—or the pharmacologically acceptable salts thereof—of general formula (1) according to claim 1 as medicaments.

10. Pharmaceutical preparations containing as active substance one or more compounds of general formula (1) according to claim 1 or the pharmacologically acceptable salts thereof, optionally in combination with conventional excipients and/or carriers.

11. A method of treating or preventing cancer or infection in a warm blood animal comprising administering an effective amount of one or more compounds of general formula (1) 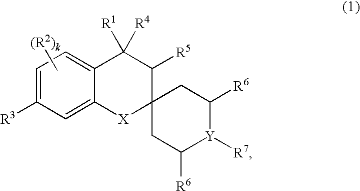
wherein
X denotes-O—, —S—, —SO— or —SO₂— and
Y denotes N or CH,
R¹ is selected from among C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl, all the above-mentioned groups optionally being substituted by one or more identical or different R^{a} and/or R^{b},
R² and R³ are each independently of one another selected from among R^{a} and R^{b}, or
R³ together with an adjacent R² in the ortho position and the two carbon atoms to which R² and R³ are fixed, may form a phenyl ring, a 5-6 membered heteroaryl, 5-7 membered cycloalkyl or 5-7 membered heterocycloalkyl, while the above-mentioned ring systems may optionally be substituted by one or more identical or different R^{a} and/or R^{b},
R⁴ denotes hydrogen, C₁₋₆alkyl or C₁₋₆haloalkyl, optionally substituted by one or more identical or different groups —OR¹ and/or —NR^{h}R^{h},
R⁵ is selected from among hydrogen, C₁₋₆haloalkyl, halogen, —CN, —C(O)OR^{h}, —C(O)NR^{h}R^{h} and C₁₋₆alkyl, the latter optionally being substituted by one or more identical or different groups —OR^{h},
each R⁶ is selected independently of one another from among hydrogen, C₁₋₆alkyl, C₆₋₁₀aryl and halogen,
R⁷, in the event that Y denotes N, is selected from among hydrogen, R^{a}, —OR^{a}, —NR^{a}R^{a}, —S(O)R^{a}, —S(O)NR^{a}R^{a}, —S(O)₂R^{a}, —S(O)₂OR^{a}, —S(O)₂NR^{a}R^{a}, —[S(O)₂]₂R^{a}, —S(O)OR^{a}, —C(O)R^{a}, —C(S)R^{a}, —N(R^{g})C(O)R^{a}, —C(NOH)R^{a}, —C(NR^{g})R^{a}, —C(O)OR^{a}, —C(O)SR³, —C(O)NR^{a}R^{a}, —C(S)NR^{a}R^{a}, —C(O)N(R^{g})NR^{a}R^{a}, —N(R^{g})C(O)NR^{a}R^{a}, —C(NR^{g})OR^{a}, —C(NR^{g})SR^{a}, —C(NR^{g})NR^{a}R^{a}, —C(O)N(R^{g})C(O)R³, —[C(O)]₂R^{a}, —[C(O)]₂OR^{a}, —[C(O)]₂NR^{a}R^{a} and —C(O)N(R^{g})C(O)OR³, or
R⁷, in the event that Y denotes CH, is selected from among 2-6 membered heteroalkyl, 5-12 membered heteroaryl, 3-14 membered heterocycloalkyl, all the above-mentioned groups optionally being substituted by one or more identical or different R^{a} and/or R^{b}, as well as —NR^{a}R^{a}, —N(OR^{a})R^{a}, —N(R^{g})NR^{a}R^{a}, —N(R^{g})S(O)R^{a}, —N(R^{g})S(O)₂R^{a}, —N[S(O)₂R^{a}]₂, —N(R^{g})S(O)₂OR^{a}, —N(R^{g})S(O)₂NR^{a}R^{a}, —N(R^{g})S(O)OR^{a}, —N(R^{g})S(O)NR^{a}R^{a}, —N(R^{g})C(O)R^{a}, —N[C(O)R^{a}]₂, —N(R^{g})C(S)R^{a}, —N[C(O)R^{a}]NR^{a}R^{a}, —N(R^{g})N(R^{g})C(O)R^{a}, —N(OR⁹)C(O)R^{a}, —N(R^{g})C(NOH)R^{a}, —N(R^{g})C(NR^{g})R^{a}, —N(R^{g})C(O)OR^{a}, —N(R^{g})C(O)SR^{a}, —N(R^{g})C(O)NR^{a}R^{a}, —N(R^{g})C(S)NR^{a}R^{a}, —N(R^{g})C(O)NR^{g}NR^{a}R^{a}, —N(R^{g})N(R^{g})C(O)NR^{a}R^{a}, N(R^{g})C(NR^{g})OR^{a}, —N(R^{g})C(NR^{g})SR^{a}, —N(R^{g})C(NR^{g})NR^{a}R^{a}, —[N(R^{g})C(O)]₂R^{a}, —N(R^{g})[C(O)]₂R^{a}, —N {[C(O)]₂R^{a}}₂, —N(R^{g})[C(O)]₂OR^{a}, —N(R^{g})[C(O)]₂NR^{a}R^{a}, —N {[C(O)]₂OR^{a}}₂, —N {[C(O)]₂NR^{a}R^{a}}₂ and —[N(R^{g})C(O)]₂OR^{a},
k denotes either 0, 1, 2 or 3,
each R^{a} independently of one another denotes hydrogen or a group optionally substituted by one or more identical or different R^{b} and/or R^{c}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{b} denotes a suitable group and is selected independently of one another from among ═O, —OR^{c}, C₁₋₃haloalkyloxy, —OCF₃, ═S, —SR^{c}, ═NR^{c}, ═NOR^{c}, ═NNR^{c}R^{c}, ═NN(R^{g})C(O)NR^{c}R^{c}, —NR^{c}R^{c}, —ONR^{c}R^{c}, —N(OR^{c})R^{c}, —N(R^{g})NR^{c}R^{c}, halogen, —CF₃, —CN, —NC, —OCN, —SCN, —NO, —NO₂, ═N₂, —N₃, —S(O)R^{c}, —S(O)OR^{c}, —S(O)₂R^{c}, —S(O)₂OR^{c}, —S(O)NR^{c}R^{c}, —S(O)₂NR^{c}R^{c}, —OS(O)R^{c}, —OS(O)₂R^{c}, —OS(O)₂OR^{c}, —OS(O)NR^{c}R^{c}, —OS(O)₂NR^{c}R^{c}, —C(O)R^{c}, —C(O)OR^{c}, —C(O)SR^{c}, —C(O)NR^{c}R^{c}, —C(O)N(R^{g})NR^{c}R^{c}, —C(O)N(R^{g})OR^{c}, —C(NR^{g})NR^{c}R^{c}, —C(NOH)R^{c}, —C(NOH)NR^{c}R^{c}, —OC(O)R^{c}, —OC(O)OR^{c}, —OC(O)SR^{c}, —OC(O)NR^{c}R^{c}, —OC(NR^{g})NR^{c}R^{c}, —SC(O)R^{c}, —SC(O)OR^{c}, —SC(O)NR^{c}, —SC(NR^{g})NR^{c}R^{c}, —N(R^{g})C(O)R^{c}, —N[C(O)R^{c}]₂, —N(OR^{g})C(O)R^{c}, —N(R^{g})C(NR^{g})R^{c}, —N(R^{g})N(R^{g})C(O)R^{c}, —N[C(O)R^{c}]NR^{c}R^{c}, —N(R^{g})C(S)R^{c}, —N(R^{g})S(O)R^{c}, —N(R^{g})S(O)OR^{c}, —N(R^{g})S(O)₂R^{c}, —N[S(O)₂R^{c}]₂, —N(R^{g})S(O)₂OR^{c}, —N(R^{g})S(O)₂NR^{c}R^{c}, —N(R^{g}) [S(O)₂]₂R^{c}, —N(R^{g})C(O)OR^{c}, —N(R^{g})C(O)SR^{c}, —N(R^{g})C(O)NR^{c}R^{c}, —N(R^{g})C(O)NR^{g}NR^{c}R^{c}, —N(R^{g})N(R^{g})C(O)NR^{c}R^{c}, —N(R^{g})C(S)NR^{c}R^{c}, —[N(R^{g})N(R^{g})C(O)]₂R^{c}, —N {[C(O)]₂R^{c}}₂, —N(R^{g})[C(O)]₂OR^{c}, —N(R^{g})[C(O)]₂NR^{c}R^{c}, —N {[C(O)]₂OR^{c}}₂, —N {[C(O)]₂NR^{c}R^{c}}₂, —[N(R^{g})C(O)]₂OR^{c}, —N(R^{g})C(NR^{g})OR^{c}, —N(R^{g})C(NOH)R^{c}, —N(R^{g})C(NR^{g})SR^{c} and —N(R^{g})C(NR^{g})NR^{c}R^{c},
each R^{c} independently of one another denotes hydrogen or a group optionally substituted by one or more identical or different R^{d} and/or R^{e}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{d} denotes a suitable group and is selected independently of one another from among ═O, —OR^{e}, C₁₋₃haloalkyloxy, —OCF₃, ═S, —SR^{e}, ═NR^{e}, ═NOR^{e}, ═NNR^{e}R^{e}, ═NN(R^{g})C(O)NR^{e}R^{e}, —ONR^{e}R^{e}, —N(R^{g})NR^{e}R^{e}, halogen, —CF₃, —CN, —NC, —OCN, —SCN, —NO, —NO₂, ═N₂, —N₃, —S(O)R^{e}, —S(O)OR^{e}, —S(O)₂R^{e}, —S(O)₂OR^{e}, —S(O)NR^{e}R^{e}, —S(O)₂NR^{e}R^{e}, —OS(O)R^{e}, —OS(O)₂R^{e}, —OS(O)₂OR^{e}, —OS(O)NR^{e}R^{e}, —OS(O)₂NR^{e}R^{e}, —C(O)R^{e}, —C(O)OR^{e}, —C(O)SR^{e}, —C(O)NR^{e}R^{e}, —C(O)N(R^{g})NR^{e}R^{e}. —C(O)N(R^{g})OR^{e}, —C(NR^{g})NR^{e}R^{e}, —C(NOH)R^{e}, —C(NOH)NR^{e}R^{e}, —OC(O)R^{e}, —OC(O)OR^{e} OC(O)SR^{e}, —OC(O)NR^{e}R^{e}, —OC(NR^{g})NR^{e}R^{e}, —SC(O)R^{e}, —SC(O)OR^{e}, —SC(O)NR^{e}R^{e}, —SC(NR^{g})NR^{e}R^{e}, —N(R^{g})C(O)R^{e}, —N[C(O)R^{e}]₂, —N(OR^{g})C(O)R^{e}, —N(R^{g})C(R^{g})R^{e}, —N(R^{g})N(R^{g})C(O)R^{e}, —N[C(O)R^{e}]NR^{e}R^{e}, —N(R^{g})C(S)R^{e}, —N(R^{g})S(O)R^{e}, —N(R^{g})S(O)OR^{e}— N(R^{g})S(O)₂R^{e}, —N[S(O)₂R^{e}]₂, —N(R^{g})S(O)₂OR^{e}, —N(R^{g})S(O)₂NR^{e}R^{e}, —N(R^{g})[S(O)₂]₂R^{e}, —N(R^{g})C(O)OR^{e}, —N(R^{g})C(O)SR^{e}, —N(R^{g})C(O)NR^{e}R^{e}, —N(R^{g})C(O)NR^{g}NR^{e}R^{e}, —N(R^{g})N(R^{g})C(O)NR^{e}R^{e}, —N(R^{g})C(S)NR^{e}R^{e}, —[N(R^{g})C(O)]₂R^{e}, —N(R^{g}) [C(O)]₂R^{e}, —N {[C(O)]₂R^{e}}₂, —N(R^{g})[C(O)]₂—R^{e}, —N(R^{g})[C(O)]₂NR^{e}R^{e}, —N{[C(O)]₂OR^{e}}₂, —N {[C(O)]₂NR^{e}R^{e}}₂, —[N(R^{g})C(O)]₂OR^{e}, —N(R^{g})C(NR^{g})OR^{e}, —N(R^{g})C(NOH)R^{e}, —N(R^{g})C(NR^{g}) SR^{e} and —N(R^{g})C(NR^{g})NR^{e}R^{e},
each R^{e} independently of one another denotes hydrogen or a group optionally substituted by one or more identical or different R^{f} and/or R^{g}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{f} denotes a suitable group and is selected independently of one another from among ═O, —OR^{g}, C₁₋₃haloalkyloxy, —OCF₃, ═S, —SR^{g}, ═NR^{g}, ═NOR^{g}, ═NNR^{g}R^{g}, ═NN(R^{e})C(O)NR^{g}R^{g}, —NR^{g}R^{g}, —ONR^{g}R^{g}, —N(R^{e})NR^{g}R^{g}, halogen, —CF₃, —CN, —NC, —OCN, —SCN, —NO, —NO₂, ═N₂, —N₃, —S(O)R^{g}, —S(O)OR^{g}, —S(O)₂R^{g}, —S(O)₂OR^{g}, —S(O)NR^{g}R^{g}, —S(O)₂NR^{g}R^{g}, —OS(O)R^{g}, —OS(O)₂R^{g}, —OS(O)₂OR^{g}, —OS(O)NR^{g}R^{g}, —OS(O)₂NR^{g}R^{g}, —C(O)R^{g}, —C(O)OR^{g}, —C(O)SR^{g}, —C(O)NR^{g}R^{g}, —C(O)N(R^{h})NR^{g}R^{g}, —C(O)N(R^{h})OR^{g}, —C(NR^{h})NR^{g}R^{g}, —C(NOH)R^{g}, —C(NOH)NR^{g}R^{g}, —OC(O)R^{g}, —OC(O)OR^{g}, —OC(O)SR^{g}, —OC(O)NR^{g}R^{g}, —OC(NR^{h})NR^{g}R^{g}, —SC(O)R^{g}, —SC(O)OR^{g}, —SC(O)NR^{g}R^{g}, —SC(NR^{h})NR^{g}R^{g}, —N(R^{h})C(O)R^{g}, —N[C(O)R^{g}]₂, —N(OR^{h})C(O)R^{g}, —N(R^{h})C(NR^{h})R^{g}, —N(R^{h})N(R^{h})C(O)R^{g}, —N[C(O)R^{g}]NR^{g}R^{g}, —N(R^{h})C(S)R^{g}, —N(R^{h})S(O)R^{g}, —N(R^{h})S(O)OR^{g}, —N(R^{h})S(O)₂R^{g}, —N[S(O)₂R^{g}]₂, —N(R^{h})S(O)₂OR^{g}, —N(R^{h})S(O)₂NR^{g}R^{g}, —N(R^{h})[S(O)₂]₂R^{g}, —N(R^{h})C(O)OR^{g}, —N(R^{h})C(O)SR^{g}, —N(R^{h})C(O)NR^{g}R^{g}, —N(R^{h})C(O)NR^{h}NR^{g}R^{g}, —N(R^{h})N(R^{h})C(O)NR^{g}R^{g}, —N(R^{h})C(S)NR^{g}R^{g}, —[N(R^{h}) C(O)]₂R^{g}, —N {[C(O)]₂R^{g} }₂, —N(R^{h})[C(O)]₂OR^{g}, —N(R^{h})[C(O)]₂NR^{g}R^{g}, —N {[C(O)]₂OR^{g}}₂, —N {[C(O)]₂NR^{g}R^{g} }₂, —[N(R^{h})C(O)]₂OR^{g}, —N(R^{h})C(NR^{h})OR^{g}, —N(R^{h})C(NOH)R^{g}, —N(R^{h})C(NR^{h})SR^{g} and —N(R^{h})C(NR^{h})NR^{g}R^{g},
each R^{g} independently of one another denotes hydrogen or a group optionally substituted by one or more identical or different R^{h}, selected from among C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered hetero-aryl, 6-18 membered heteroarylalkyl, 3-14 membered heterocycloalkyl and 4-14 membered heterocycloalkylalkyl,
each R^{h} is selected independently of one another from among hydrogen, C₁₋₆alkyl, 2-6 membered heteroalkyl, C₁₋₆haloalkyl, C₃₋₁₀cycloalkyl, C₄₋₁₆cycloalkylalkyl, C₆₋₁₀aryl, C₇₋₁₆arylalkyl, 5-12 membered heteroaryl, 6-18 membered heteroarylalkyl, 3-14 membered heterocycloalkyl and 4-14 membered heterocycloalkylalkyl,
optionally in the form of the tautomers, the racemates, the enantiomers, the diastereomers and the mixtures thereof, and optionally the pharmacologically acceptable salts thereof,
for preparing a pharmaceutical composition for the treatment and/or prevention of cancer and infectious diseases.

12. (canceled)

13. Pharmaceutical preparation comprising a compound of general formula (1) according to claim 1 and at least one other cytostatic or cytotoxic active substance, different from formula (1), optionally in the form of the tautomers, the racemates, the enantiomers, the diastereomers and the mixtures thereof, and optionally the pharmacologically acceptable salts thereof.
